(19) United States
(12) Patent Application Publication
(10) Pub. No.: US 2015/0352034 A1
(43) Pub. Date: Dec. 10, 2015

(54) Personal Care Products Containing Microalgae or Extracts Thereof

(71) Applicant: Solazyme, Inc., South San Francisco, CA (US)

(72) Inventors: Celine Schiff-Deb, South San Francisco, CA (US); Suruchi Sharma, South San Francisco, CA (US)

(21) Appl. No.: 14/733,076

(22) Filed: Jun. 8, 2015

Related U.S. Application Data
(60) Provisional application No. 62/009,296, filed on Jun. 8, 2014.
(60) Provisional application No. 62/082,026, filed on Nov. 19, 2014.

(51) Int. Cl. A61K 8/92; A61Q 5/00

(52) U.S. Cl. CPC A61K 8/92; A61Q 5/00; A61K 2800/70

(57) ABSTRACT

Compositions and methods for their use as personal care products are disclosed. The compositions contain microalgae or microalgal components and are beneficial for use in skin and hair care.

### CROSS REFERENCE TO RELATED APPLICATIONS

This application claims the benefit under 35 U.S.C. 119(e) of U.S. Provisional Patent Application No. 62/009,296, filed Jun. 8, 2014 and U.S. Provisional Patent Application No. 62/082,026, filed Nov. 19, 2014, each of which is incorporated herein by reference in its entirety.

### BACKGROUND

Consumer products for effectively maintaining or promoting healthy skin and hair are highly sought after. Skin and hair care products that can simultaneously prevent, improve, or reverse the effects of aging, ultraviolet radiation, and water loss are especially desirable, as are those that can also impart a luxurious feel and are absorbent and non-greasy.

### SUMMARY

In an embodiment, the present invention provides compositions and methods for their use as personal care products. In some embodiments the compositions are formulated for topical administration. The compositions can contain cosmetic ingredients suitable for human use and are compatible with the microalgae or microalgal components provided herein.

In one embodiment, provided is a method for preventing or treating ultraviolet radiation damage to human skin or hair, the method comprising applying to the human skin or hair an effective amount of a topical composition comprising one or more of a microalgae, an extract thereof, or a modified extract thereof, wherein the composition reduces thymine dimer formation and/or increases cell viability in the skin or hair.

In one embodiment, provided is a method for increasing or controlling hydration in human skin or hair, the method comprising applying to the human skin or hair an effective amount of a topical composition comprising one or more of a microalgae, an extract thereof, or a modified extract thereof, wherein the composition increases hydration and/or limits water loss in the skin or hair.

In one embodiment, provided is a method for improving appearance of skin in a human, the method comprising applying to the human skin an effective amount of a topical composition comprising one or more of a microalgae, an extract thereof, or a modified extract thereof, wherein the composition increases one or more of hyaluronic acid, collagen, or elastin in the skin. In some embodiments, the improvement in appearance comprises an improvement in one or more of fine lines, wrinkles, firmness, smoothness, softness, tone, radiance, luster, brightness, color, thickness, elasticity, or resiliency.

In one embodiment, provided is a method for introducing or modifying a sensory property of a personal care composition, the method comprising blending a microalgae, extract thereof, or modified extract with the composition, wherein the sensory property is one or more of slipperiness, silkiness, absorbency, spreadability, moisture, or increased lather.

In some embodiments, the composition is an emollient, a moisturizer, a cream, a liniment, a lotion, a soap, a balm, a shampoo, a hair conditioner, a hair mask, a skin oil, a hair oil, an ointment, a makeup, a sun care product, or a baby product. In some embodiments, the composition is a shaving cream, hand cream, or eye cream.

In some embodiments, the composition is a skin toner. In some embodiments, the skin toner is applied after cleaning the skin to restore lost nutrients.

In some embodiments, the composition is a nail care product. In other embodiments, the composition is a nail cream.

In some embodiments, the composition is a makeup remover. In some embodiments, the makeup remover comprises a cell oil produced by a microalgae. In other embodiments, the makeup remover is a wipe or cream. In still other embodiments, the makeup remover is a mascara remover. In other embodiments, the makeup remover is a cold cream.

In some embodiments, the compositions according to the present invention include, for example, skin cleaners such as soap, cleansing creams, cleansing lotions, cleansing milks, cleansing pads, facial washes, and body shampoos. In some cases, the composition is a cleansing oil or a face oil.

In some embodiments, the compositions provided herein when applied to hair increases one or more of shine, combability, hair strength, resistance to UV damage, resistance to pollution damage, resistance to moisture loss, and resistance to split ends. In some embodiments, the makeup remover comprises a cell oil produced by a microalgae.

In some embodiments, hair treated with a composition provided herein has one or more properties of:

a) reduced entanglements;

b) reduced snagging;

c) reduced frizz;

d) reduced split ends;

e) less force required for brushing or combing;

f) reduced breakage;

g) increased resistance to heat damage such as from blow drying or hair irons; or

h) increased shine

as compared to untreated hair.

In some embodiments, hair treated with a composition provided herein prevents or retards the progression of hair loss.

In some embodiments, the composition is an exfoliant. The exfoliant is useful for removing skin cells such as dead skin cells, body oil, makeup, dirt, bacteria, and pore-occluding materials. In some embodiments the exfoliant is a facial scrub or a body scrub. In other embodiments the exfoliant can also be used in a facial mask or exfoliating serum. The exfoliant can be used to treat dry skin, psoriasis, rosacea, eczema, acne, acne scars, clogged pores (blackheads and whiteheads), and other skin conditions. The exfoliant can also have a moisturizing effect that eliminates the need to apply a separate moisturizer after use of the exfoliant. Use of the exfoliant compositions provided herein can be beneficial in allowing for one or more of the following features: cleaner skin, smoother skin, softer skin, improved complexion, improved luster, and greater resistance to skin blemishes and breakouts such as dry and/or colored patches, skin rashes, acne, blackheads, and whiteheads. In still other embodiments, the exfoliant compositions provided herein have an anti-microbial effect and can be used as a wound cleanser.

In some embodiments, provided is a method for exfoliating skin, the method comprising:

a) providing an exfoliant composition comprising microalgal cells, the cells comprising at least 10% oil by dry cell weight;
b) applying the exfoliant composition to the skin; and
c) rubbing the exfoliant composition against the skin, wherein friction between the microalgal cells and the skin removes at least the outermost layer of skin.

The stratum corneum is the outer layer of dead skin cells and is typically 15-20 layers thick (10-20 micron thickness). In some embodiments of the exfoliant compositions and methods, at least two or more of the outermost skin layers are removed. In other embodiments, at least three or more of the outermost skin layers are removed. In other embodiments, at least four or more of the outermost skin layers are removed. In still other embodiments, up to 10 of the outermost skin layers are removed. In some embodiments, the exfoliant composition is rubbed against the skin in one or more of a circular, twisting, unidirectional, or back and forth motion. Following exfoliation, the composition can be wiped or rinsed from the skin such as with warm water.

In some embodiments, the exfoliant compositions comprise microalgal cells, the cells comprising at least 10% oil by dry cell weight. In some embodiments, the cells comprise at least 20%, 40%, 50%, 60%, 80%, 85%, or 90% oil by dry cell weight. In some embodiments, the exfoliant compositions comprise microalgal cells, the cells comprising at least 60% oil by dry cell weight.

In some embodiments, the cells comprise 1% to 95% by weight of the exfoliant composition. In other embodiments, the cells comprise at least 2%, 5%, 7%, 10%, 20%, 30%, 40%, 50%, 60%, 70%, or 80% by weight of the exfoliant composition.

In still other embodiments, the exfoliant compositions further comprise a microalgal triglyceride oil or a fatty acid alkyl ester derived from a microalgal triglyceride oil or a combination thereof. In other embodiments, the alkyl ester is a fatty acid methyl ester or a fatty acid ethyl ester. In some embodiments, the microalgal triglyceride oil or a fatty acid alkyl ester derived from a microalgal triglyceride oil, or a combination thereof, comprise at least 1%, 2%, 5%, 7%, 10%, 20%, 30%, 40%, 50%, 60%, 70%, or 80% by weight of the exfoliant composition.

In some embodiments, the exfoliant compositions comprise at least 1% microalgal cells and at least 10% of a microalgal triglyceride oil or a fatty acid alkyl ester derived from a microalgal triglyceride oil. In some embodiments, the exfoliant compositions comprise at least 1% microalgal cells and at least 25% of a microalgal triglyceride oil or a fatty acid alkyl ester derived from a microalgal triglyceride oil.

In some embodiments, the exfoliant compositions comprise 10% to 70% microalgal cells and 30% to 90% of a microalgal triglyceride oil or a fatty acid alkyl ester derived from a microalgal triglyceride oil. In some embodiments, the exfoliant compositions comprise 10% to 50% microalgal cells and 50% to 90% of a microalgal triglyceride oil or a fatty acid alkyl ester derived from a microalgal triglyceride oil.

In some embodiments, the exfoliant compositions further comprise a silicon elastomer.

The microalgal cells provided herein can adhere to each other and agglomerate into larger particles. The particles sizes can be selected by passing the particles through a size selective screen such as a wire mesh. In some embodiments of the exfoliant compositions provided herein, the average particle sizes of the agglomerated microalgal cells are less than 0.5 mm, less than 1 mm, between 1 and 2 mm, or greater than 2 mm.

In some embodiments, the microalgae is a whole cell or a lysed cell.

In some embodiments, the extract comprises a triglyceride. In other embodiments, the extract is an aqueous extract. In still other embodiments, the extract comprises a polysaccharide.

In some embodiments, the modified extract is a fatty acid derivative. In other embodiments, the modified extract is a fatty acid alkyl ester. In still other embodiments, the fatty acid alkyl ester is a methyl, ethyl, or isopropyl fatty acid ester.

In some embodiments, the composition comprises at least 1% by weight of the microalgae, extract thereof, modified extract thereof, or a combination thereof.

In some embodiments, the microalgae is of the genus Prototheca, Auxenochlorella, Chlorella, or Parachlorella. In other embodiments, the microalgae is of the species Prototheca moriformis.

In some embodiments, the microalgae is of the species Chlorella (Auxeochlorella) protothecoides.

In some embodiments, the composition comprises a cell oil produced by the microalgae and/or encapsulated in the microalgae, wherein the microalgae is Chlorella (Auxeochlorella) protothecoides and the oil has a fatty acid profile of greater than 15% C16:0 and greater than 55% 18:1.

In some embodiments, the composition comprises a cell oil produced by the microalgae and/or encapsulated in the microalgae, the oil having a fatty acid profile of greater than 50%, 60%, 70%, or 80% combined C10:0 and C12:0. In other embodiments, the oil has a fatty acid profile of greater than 80% combined C10:0 and C12:0.

In some embodiments, the composition comprises a cell oil produced by the microalgae and/or encapsulated in the microalgae, the oil having a fatty acid profile of greater than 60% C10:0 and C12:0 and greater than 10% C14:0.

In some embodiments, the composition comprises a cell oil produced by the microalgae and/or encapsulated in the microalgae, the oil having a fatty acid profile of greater than 40%, 45%, or 50% C14:0. In other embodiments, the oil has a fatty acid profile of greater than 50% C14:0.

In some embodiments, the composition comprises a cell oil produced by the microalgae and/or encapsulated in the microalgae, the oil having a fatty acid profile of greater than 85% C18:1 and less than 3% polyunsaturates.

In some embodiments, the composition comprises a cell oil produced by the microalgae and/or encapsulated in the microalgae, the oil having a fatty acid profile of at least 70% SOS and no more than 4% trisaturates.

In some embodiments, the composition comprises a cell oil produced by the microalgae and/or encapsulated in the microalgae, the oil having a fatty acid profile of greater than 50% C18:0 and greater than 30% C18:1.

These and other embodiments and features of the invention are further described in the following drawings and detailed description.

### BRIEF DESCRIPTION OF THE DRAWINGS

FIG. 1 shows an MTT assay measuring the viability of cells treated with various test oils as illustrated in the Examples.

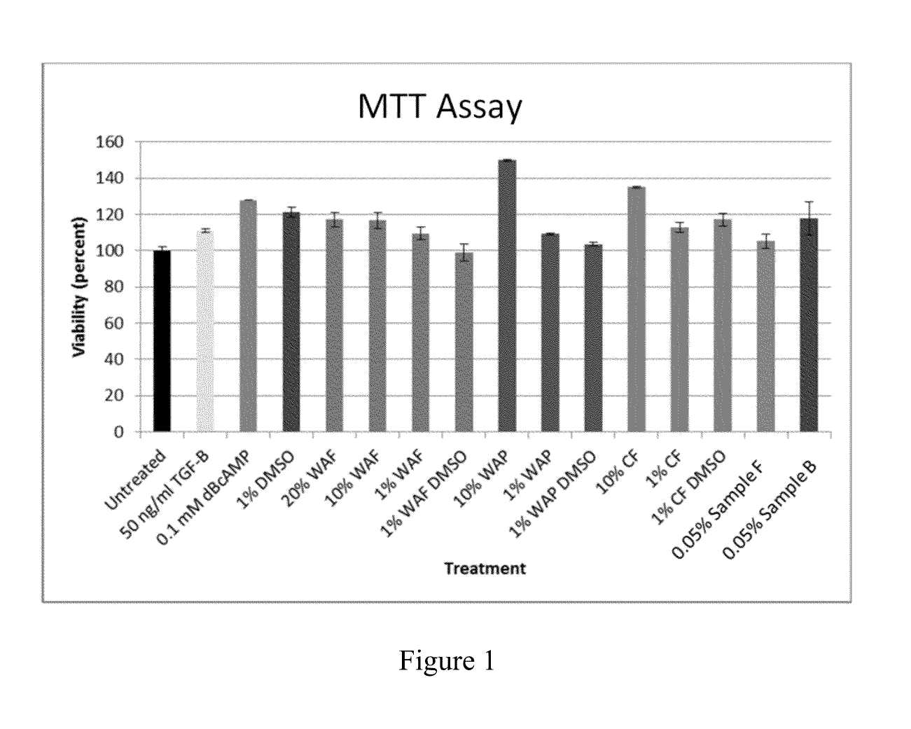

FIG. 2 shows a Type I Collagen assay measuring concentrations of type I C-peptide fragments to quantify collagen production in tissue treated with various test oils as illustrated in the Examples.

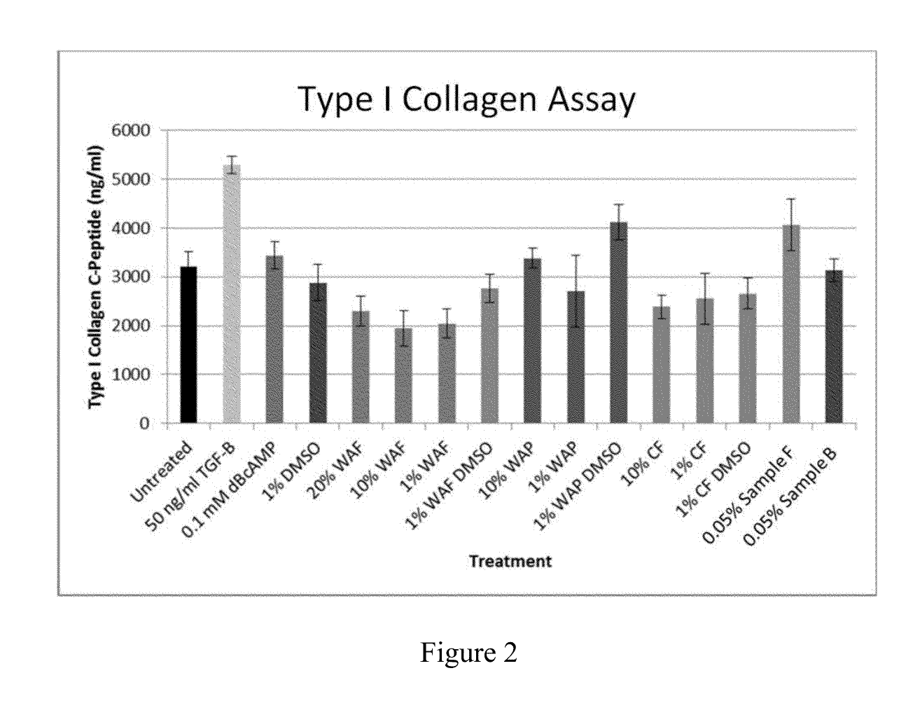

FIG. 3 shows an elastin assay measuring for elastin production in tissue treated with various test oils illustrated in the Examples.

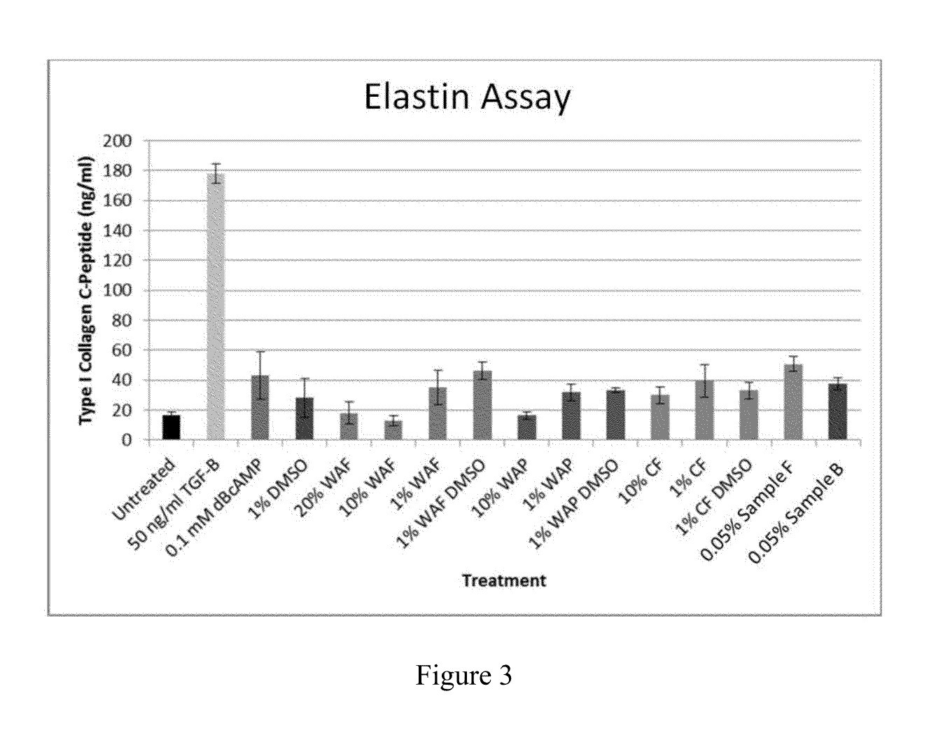

FIG. 4 shows a hyaluronic acid assay measuring concentrations of hyaluronic acid in tissues treated with various test oils illustrated in the Examples.

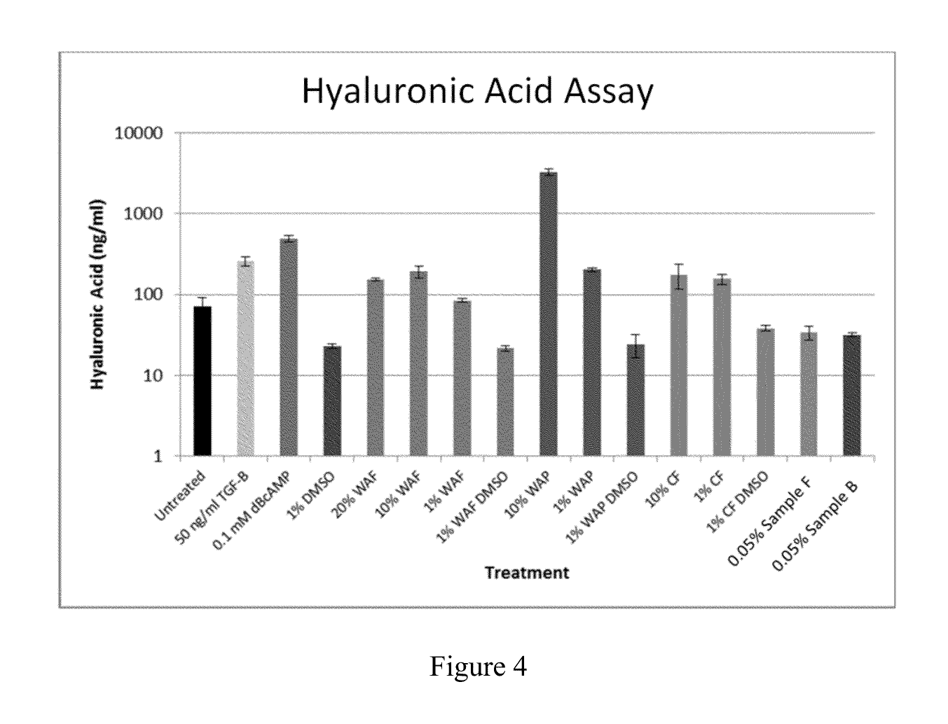

FIG. 5 shows an MTT assay measuring the viability of cells treated with various test oils after prolonged exposure to UVB radiation illustrated in the Examples.

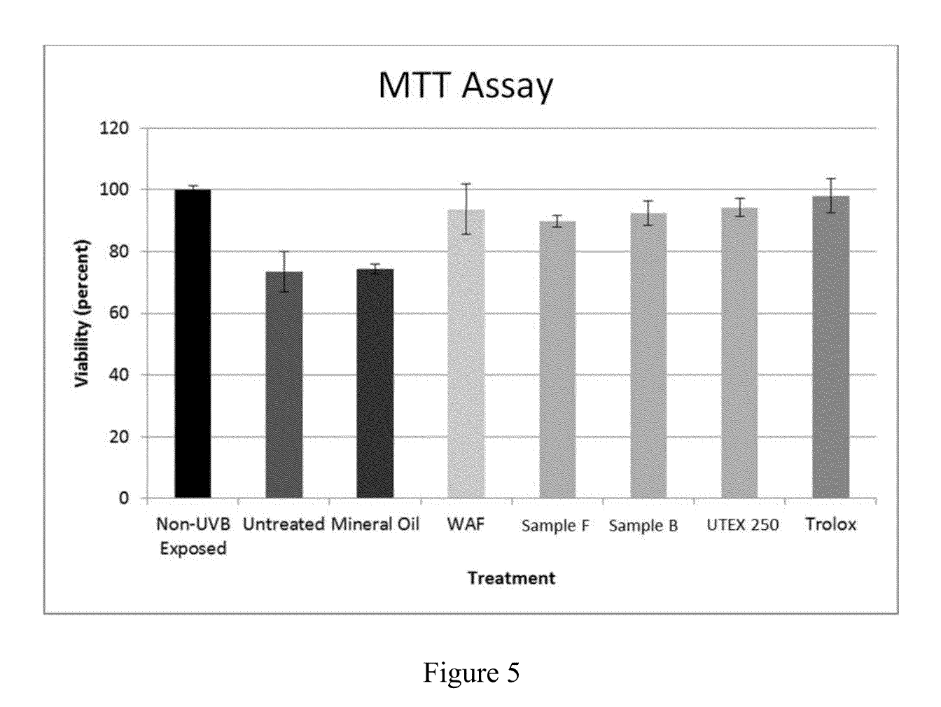

FIG. 6 shows a TT Dimer assay measuring the concentration of TT Dimer formed in cells exposed to UVB radiation after treatment with various test oils illustrated in the Examples.

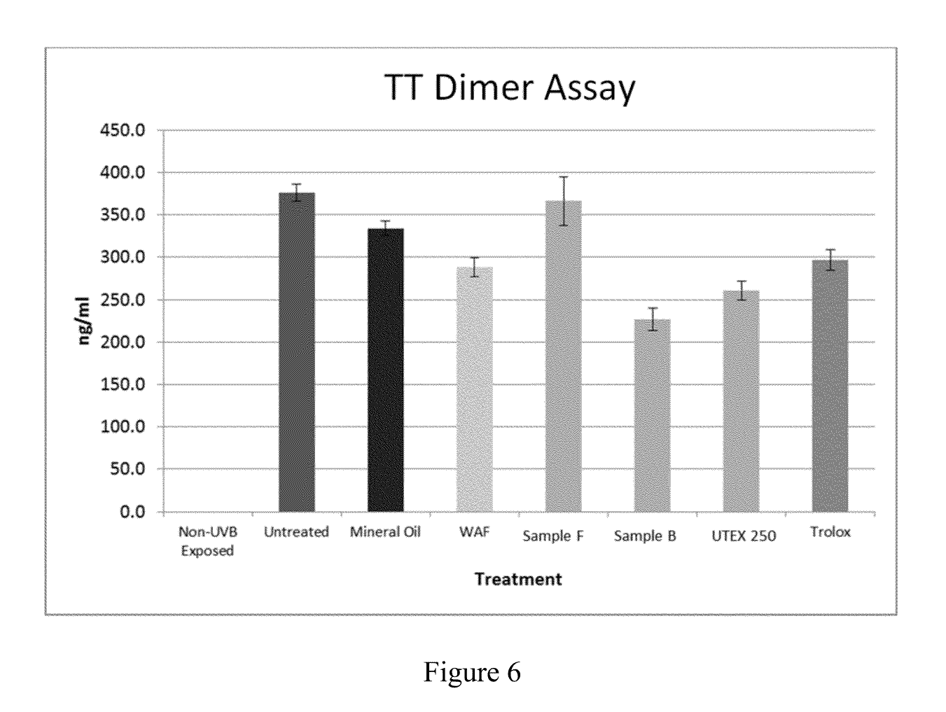

### DETAILED DESCRIPTION

### Definitions

“Cosmetic ingredient” refers to an ingredient conventionally used in cosmetic products that is not physically or chemically incompatible with the microalgal components described herein. “Cosmetic ingredients” include, without limitation, absorbents, abrasives, anticaking agents, antifoaming agents, antimicrobial agents, binders, biological additives, buffering agents, bulking agents, chemical additives, cosmetic biocides, denaturants, cosmetic astringents, drug astringents, external analgesics, film formers, humectants, opacifying agents, fragrances, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins and derivatives thereof, and natural extracts. Such “cosmetic ingredients” are known in the art. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993).

The “effective amount” is the amount necessary to bring about a desired effect. Such effects include an improvement in appearance, UV protection, anti-aging effects, hydration, and effectiveness as a skin barrier. The effects can be measured by standard assays used in the cosmetic industry including those disclosed herein. For example, cell based assays can measure levels of collagen, elastin, and hyaluronic acid, which are important components in skin health and repair and whose levels are found to be depressed in damaged or aging skin.

“Ultraviolet radiation” refers to electromagnetic radiation in the ultraviolet wavelength range. Ultraviolet radiation can come from the sun and includes UV-A (320-400 nm) and UV-B (290-320 nm) radiation. “Ultraviolet radiation damage” includes any deleterious effects caused by exposure to UV radiation including sunburn, discoloration, uneven skin tone, age spots, wrinkles, freckles, DNA damage, DNA mutations, and skin cancer. DNA damage includes disruption of DNA base paring and formation of pyridine dimers such as thymine dimers. These dimers, if left unrepaired, can lead to uncontrollable DNA replication and melanomas.

“Microalgae” refers to eukaryotic microbial organisms that contain a chloroplast or other plastid, and optionally that are capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source. Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis. Examples of obligate heterotrophs include certain dinoflagellate algae species and species of the genus Prototheca. Microalgae include those belonging to the phylum Chlorophyta and in the class Trebouxiophyceae. Within this class are included microalgae belonging to the order Chlorellales, optionally the family Chlorellaceae, and optionally the genus Prototheca, Auxenochlorella, Chlorella, or Parachlorella.

“Microalgal extracts” refer to any cellular components that are extracted from the cell or are secreted by the cells. The extracts include those can be obtained by mechanical pressing of the cells or by solvent extraction. Cellular components can include, but are not limited to, microalgal oil, proteins, carbohydrates, phospholipids, polysaccharides, macromolecules, minerals, cell wall, trace elements, carotenoids, and sterols. In some cases the extract is a polysaccharide that is secreted from a cell into the extracellular environment and has lost any physical association with the cells. In other cases the polysaccharide remain associated with the cell wall. Polysaccharides are typically polymers of monosaccharide units and have high molecular weights, usually with an average of 2 million Daltons or greater, although fragments can be smaller in size.

“Microalgal oils” or “cell oils” refer to lipid components produced by microalgal cells such as triglycerides.

“Modified microalgal extracts” refer to extracts that are chemically or enzymatically modified. For example, triglyceride extracts can be converted to fatty acid alkyl esters (e.g. fatty acid methyl esters) by transesterification.

“Microalgal biomass,” “algal biomass” or “biomass” refers to material produced by growth and/or propagation of microalgal cells. Biomass may contain cells and/or intracellular contents as well as extracellular material. Extracellular material includes, but is not limited to, compounds secreted by a cell.

“Dry weight” or “dry cell weight” refer to weight as determined in the relative absence of water. For example, reference to a component of microalgal biomass as comprising a specified percentage by dry weight means that the percentage is calculated based on the weight of the biomass after all or substantially all water has been removed.

“Exogenous gene” refers to a nucleic acid transformed into a cell. A transformed cell may be referred to as a recombinant cell, into which additional exogenous gene(s) may be introduced. The exogenous gene may be from a different species (and so heterologous), or from the same species (and so homologous) relative to the cell being transformed. In the case of a homologous gene, it occupies a different location in the genome of the cell relative to the endogenous copy of the gene. The exogenous gene may be present in more than one copy in the cell. The exogenous gene may be maintained in a cell as an insertion into the genome or as an episomal molecule.

“Exogenously provided” describes a molecule provided to the culture media of a cell culture.

“Fixed carbon source” means molecule(s) containing carbon, preferably organic, that are present at ambient temperature and pressure in solid or liquid form.

“Fatty acid profile” refers to the distribution of different carbon chain lengths and saturation levels of fatty acid moieties in a particular sample of biomass or oil. “Triglycerides” are lipids where three fatty acid moieties are attached to a glycerol moiety. A sample could contain lipids in which approximately 60% of the fatty acid moieties is C18:1, 20% is C18:0, 15% is C16:0, and 5% is C14:0. In cases in which a carbon length is referenced generically, such as “C18”, such reference can include any amount of saturation; for example, microalgal biomass that contains 20% lipid as C18 can include C18:0, C18:1, C18:2, and the like, in equal or varying amounts, the sum of which constitute 20% of the biomass.

“Good Manufacturing Practices” (GMP) refers to the regulations promulgated by the US Food and Drug Association under the authority of Food, Drug and Cosmetics Act that require manufacturers to take precautions to insure that their products are safe, pure and effective. Chapter VI of the FD&C (21 U.S.C. 361) covers regulations related to cosmetics.

“Lipids” are a class of molecules that are soluble in nonpolar solvents (such as ether and hexane) and are relatively or completely insoluble in water. Lipid molecules have these properties because they consist largely of long hydrocarbon tails which are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); nonglycerides (sphingolipids, tocopherols, tocotrienols, sterol lipids including cholesterol and steroid hormones, prenol lipids including terpenoids, fatty alcohols, waxes, and polyketides); and complex lipid derivatives (sugar-linked lipids, or glycolipids, and protein-linked lipids).

“Homogenate” means biomass that has been physically disrupted.

“Homogenize” means to blend two or more substances into a homogenous or uniform mixture. In some embodiments, a homogenate is created. In other embodiments, the biomass is predominantly intact, but homogeneously distributed throughout the mixture.

“Predominantly intact cells” refers to a population of cells which comprise more than 50%, 75%, or 90% intact cells. “Intact” refers to the physical continuity of the cellular membrane enclosing the intracellular components of the cell and means that the cellular membrane has not been disrupted in any manner that would release the intracellular components of the cell to an extent that exceeds the permeability of the cellular membrane under conventional culture conditions or those culture conditions described herein.

Reference to proportions by volume, i.e., “v/v,” means the ratio of the volume of one substance or composition to the volume of a second substance or composition. For example, reference to a composition that comprises 5% v/v microalgal oil and at least one other cosmetic ingredient means that 5% of the composition's volume is composed of microalgal oil; e.g., a composition having a volume of 100 mm³ would contain 5 mm³ of microalgal oil and 95 mm³ of other constituents.

Reference to proportions by weight, i.e., “w/w,” means the ratio of the weight of one substance or composition to the weight of a second substance or composition. For example, reference to a cosmetic composition that comprises 5% w/w microalgal biomass and at least one other cosmetic ingredient means that 5% of the cosmetic composition is composed of microalgal biomass; e.g., a 100 mg cosmetic composition would contain 5 mg of microalgal biomass and 95 mg of other constituents.

### Microalgal Cells and Extracts

The microalgal cells can be prepared and heterotrophically cultured according to methods such as those described in WO2008/151149, WO2010/063031, WO2010/045368, WO2010/063032, WO2011/150411, WO2013/158938, 61/923,327 filed Jan. 3, 2014, PCT/US2014/037898 filed May 13, 2014, and in U.S. Pat. No. 8,557,249. The microalgal cells can be wild type cells or can be modified by genetic engineering and/or classical mutagenesis to alter their fatty acid profile and/or lipid productivity or other physical properties such as color.

In some embodiments, the cell wall of the microalgae must be disrupted during the use of the cosmetic product (e.g., soaps containing whole microalgal cells) in order to release the active components. Hence, in some embodiments having strains of microalgae with cell walls susceptible to disruption are preferred. This criterion is particularly preferred when the algal biomass is to be used as whole algal cells as an ingredient in the final cosmetic production. Susceptibility to disruption of the cell wall is generally decreased for microalgal strains which have a high content of cellulose/hemicellulose in the cell walls.

In particular embodiments, the wild-type or genetically engineered microalgae comprise cells that are at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% or more oil by dry weight. Preferred organisms grow heterotrophically (on sugars in the absence of light).

In some embodiments, the microalgae is from the genus Chlorella. Chlorella is a genus of single-celled green algae, belonging to the phylum Chlorophyta. Chlorella cells are generally spherical in shape, about 2 to 10 μm in diameter, and lack flagella. Some species of Chlorella are naturally heterotrophic. In some embodiments, the microalgae used in the methods of the invention is Chlorella (auexnochlorella) protothecoides, Chlorella ellipsoidea, Chlorella minutissima, Chlorella zofinienesi, Chlorella luteoviridis, Chlorella kessleri, Chlorella sorokiniana, Chlorella fusca var. vacuolata Chlorella sp., Chlorella cf. minutissima or Chlorella emersonii. Other species of Chlorella those selected from the group consisting of anitrata, Antarctica, aureoviridis, candida, capsulate, desiccate, ellipsoidea (including strain CCAP 211/42), emersonii, fusca (including var. vacuolata), glucotropha, infusionum (including var. actophila and var. auxenophila), kessleri (including any of UTEX strains 397, 2229, 398), lobophora (including strain SAG 37.88), luteoviridis (including strain SAG 2203 and var. aureoviridis and lutescens), miniata, cf. minutissima, minutissima (including UTEX strain 2341), mutabilis, nocturna, ovalis, parva, photophila, pringsheimii, protothecoides (including any of UTEX strains 1806, 411, 264, 256, 255, 250, 249, 31, 29, 25 or CCAP 211/8D, or CCAP 211/17 and var. acidicola), regularis (including var. minima, and umbricata), reisiglii (including strain CCP 11/8), saccharophila (including strain CCAP 211/31, CCAP 211/32 and var. ellipsoidea), salina, simplex, sorokiniana (including strain SAG 211.40B), sp. (including UTEX strain 2068 and CCAP 211/92), sphaerica, stigmatophora, trebouxioides, vanniellii, vulgaris (including strains CCAP 211/11K, CCAP 211/80 and f. tertia and var. autotrophica, viridis, vulgaris, vulgaris f. tertia, vulgaris f. viridis), xanthella, and zofingiensis.

In addition to Chlorella, other genera of microalgae can also be used in the methods and compositions provided herein. In some embodiments, the microalgae is a species selected from the group consisting Parachlorella kessleri, Parachlorella beijerinckii, Neochloris oleabundans, Bracteacoccus, including B. grandis, B. cinnabarinas, and B. aerius, Bracteococcus sp. or Scenedesmus rebescens. Other nonlimiting examples of microalgae species include those species from the group of species and genera consisting of Achnanthes orientalis; Agmenellum; Amphiprora hyaline; Amphora, including A. coffeiformis including A. c. linea, A.c. punctata, A.c. taylori, A.c. tenuis, A.c. delicatissima, A.c. delicatissima capitata; Anabaena; Ankistrodesmus, including A. falcatus; Boekelovia hooglandii; Borodinella; Botryococcus braunii, including B. sudeticus; Bracteoccocus, including B. aerius, B. grandis, B. cinnabarinas, B. minor, and B. medionucleatus; Carteria; Chaetoceros, including C. gracilis, C. muelleri, and C. muelleri subsalsum; Chlorococcum, including C. infusionum; Chlorogonium; Chroomonas; Chrysosphaera; Cricosphaera; Crypthecodinium cohnii; Cryptomonas; Cyclotella, including C. cryptica and C. meneghiniana; Dunaliella, including D. bardawil, D. bioculata, D. granulate, D. maritime, D. minuta, D. parva, D. peircei, D. primolecta, D. salina, D. terricola, D. tertiolecta, and D. viridis; Eremosphaera, including E. viridis; Ellipsoidon; Euglena; Franceia; Fragilaria, including F. crotonensis; Gleocapsa; Gloeothamnion; Hymenomonas; Isochrysis, including I. aff galbana and I. galbana; Lepocinclis; Micractinium (including UTEX LB 2614); Monoraphidium, including M. minutum; Monoraphidium; Nannochloris; Nannochloropsis, including N. salina; Navicula, including N. acceptata, N. biskanterae, N. pseudotenelloides, N. pelliculosa, and N. saprophila; Neochloris oleabundans; Nephrochloris; Nephroselmis; Nitschia communis; Nitzschia, including N. alexandrina, N. communis, N. dissipata, N. frustulum, N. hantzschiana, N. inconspicua, N. intermedia, N. microcephala, N. pusilla, N. pusilla elliptica, N. pusilla monoensis, and N. quadrangular; Ochromonas; Oocystis, including O. parva and O. pusilla; Oscillatoria, including O. limnetica and O. subbrevis; Parachlorella, including P. beijerinckii (including strain SAG 2046) and P. kessleri (including any of SAG strains 11.80, 14.82, 21.11H9); Pascheria, including P. acidophila; Pavlova; Phagus; Phormidium; Platymonas; Pleurochrysis, including P. carterae and P. dentate; Prototheca, including P. stagnora (including UTEX 327), P. portoricensis, and P. moriformis (including UTEX strains 1441, 1435, 1436, 1437, 1439); Pseudochlorella aquatica; Pyramimonas; Pyrobotrys; Rhodococcus opacus; Sarcinoid chrysophyte; Scenedesmus, including S. armatus and S. rubescens; Schizochytrium; Spirogyra; Spirulina platensis; Stichococcus; Synechococcus; Tetraedron; Tetraselmis, including T. suecica; Thalassiosira weissflogii; and Viridiella fridericiana.

Microalgae Strain Lacking or that has Significantly Reduced Pigmentation

Microalgae, such as Chlorella, can be capable of either photosynthetic or heterotrophic growth. When grown in heterotrophic conditions where the carbon source is a fixed carbon source and in the absence of light, the normally green colored microalgae has a yellow color, lacking or is significantly reduced in green pigmentation. Microalgae of reduced (or lacking in) green pigmentation can be advantageous as a cosmetic ingredient. One advantage of microalgae of reduced (or is lacking in) green pigmentation is that as a cosmetic ingredient, the addition of the microalgae to cosmetics will not impart a green color that can be unappealing to the consumer. The reduced green pigmentation of microalgae grown under heterotrophic conditions is transient. When switched back to phototrophic growth, microalgae capable of both phototrophic and heterotrophic growth will regain the green pigmentation. Thus, it is advantageous to generate a microalgae strain that is capable of heterotrophic growth, so it is reduced or lacking in green pigmentation.

In some embodiments, it may be advantageous to reduce the amount of general pigmentation (whether yellow or green). One method for generating such microalgae strain lacking in or has significantly reduced pigmentation is through mutagenesis and then screening for the desired phenotype. Several methods of mutagenesis are known and practiced in the art. For example, Urano et al., (Urano et al., J Bioscience Bioengineering (2000) v. 90(5): pp. 567-569) describes yellow and white color mutants of Chlorella ellipsoidea generated using UV irradiation. Kamiya (Kamiya, Plant Cell Physiol. (1989) v. 30(4): 513-521) describes a colorless strain of Chlorella vulgaris, 11 h (M125).

In addition to mutagenesis by UV irradiation, chemical mutagenesis can also be employed in order to generate microalgae with reduced (or lacking in) pigmentation. Chemical mutagens such as ethyl methanesulfonate (EMS) or N-methyl-N′nitro-N-nitroguanidine (NTG) have been shown to be effective chemical mutagens on a variety of microbes including yeast, fungi, mycobacterium and microalgae. Mutagenesis can also be carried out in several rounds, where the microalgae is exposed to the mutagen (either UV or chemical or both) and then screened for the desired reduced pigmentation phenotype. Colonies with the desired phenotype are then streaked out on plates and reisolated to ensure that the mutation is stable from one generation to the next and that the colony is pure and not of a mixed population.

In a particular example, Chlorella protothecoides was used to generate strains lacking in or with reduced pigmentation using a combination of UV and chemical mutagenesis. Chlorella protothecoides was exposed to a round of chemical mutagenesis with NTG and colonies were screened for color mutants. Colonies not exhibiting color mutations were then subjected to a round of UV irradiation and were again screened for color mutants. In one embodiment, a Chlorella protothecoides strain lacking in pigmentation was isolated and is Chlorella protothecoides 33-55, deposited on Oct. 13, 2009 at the American Type Culture Collection at 10801 University Boulevard, Manassas, Va. 20110-2209, in accordance with the Budapest Treaty, with a Patent Deposit Designation of PTA-10397. In another embodiment, a Chlorella protothecoides strain with reduced pigmentation was isolated and is Chlorella protothecoides 25-32, deposited on Oct. 13, 2009 at the American Type Culture Collection at 10801 University Boulevard, Manassas, Va. 20110-2209, in accordance with the Budapest Treaty, with a Patent Deposit Designation of PTA-10396.

### Media and Culture Conditions for Microalgae

Microalgae are cultured in liquid media to propagate biomass. Microalgal species are grown in a medium containing a fixed carbon and/or fixed nitrogen source in the absence of light. Such growth is known as heterotrophic growth. For some species of microalgae, for example, heterotrophic growth for extended periods of time such as 10 to 15 or more days under limited nitrogen conditions results accumulation of high lipid content in cells.

Microalgal culture media typically contains components such as a fixed carbon source (discussed below), a fixed nitrogen source (such as protein, soybean meal, yeast extract, cornsteep liquor, ammonia (pure or in salt form), nitrate, or nitrate salt), trace elements (for example, zinc, boron, cobalt, copper, manganese, and molybdenum in, e.g., the respective forms of ZnCl₂, H₃BO₃, CoCl₂.6H₂O, CuCl₂.2H₂O, MnCl₂.4H₂O and (NH₄)₆Mo₇O₂₄.4H₂O), optionally a buffer for pH maintenance, and phosphate (a source of phosphorous; other phosphate salts can be used). Other components include salts such as sodium chloride, particularly for seawater microalgae.

In a particular example, a medium suitable for culturing Chlorella protothecoides comprises Proteose Medium. This medium is suitable for axenic cultures, and a 1 L volume of the medium (pH ˜6.8) can be prepared by addition of 1 g of proteose peptone to 1 liter of Bristol Medium. Bristol medium comprises 2.94 mM NaNO₃, 0.17 mM CaCl₂.2H₂O, 0.3 mM MgSO₄.7H₂O, 0.43 mM, 1.29 mM KH₂PO₄, and 1.43 mM NaCl in an aqueous solution. For 1.5% agar medium, 15 g of agar can be added to 1 L of the solution. The solution is covered and autoclaved, and then stored at a refrigerated temperature prior to use. Other methods for the growth and propagation of Chlorella protothecoides to high oil levels as a percentage of dry weight have been described (see for example Miao and Wu, J. Biotechnology, 2004, 11:85-93 and Miao and Wu, Biosource Technology (2006) 97:841-846 (demonstrating fermentation methods for obtaining 55% oil dry cell weight)). High oil algae can typically be generated by increasing the length of a fermentation while providing an excess of carbon source under nitrogen limitation.

Solid and liquid growth media are generally available from a wide variety of sources, and instructions for the preparation of particular media that is suitable for a wide variety of strains of microorganisms can be found, for example, online at a site maintained by the University of Texas at Austin for its culture collection of algae (UTEX). For example, various fresh water media include 1/2, 1/3, 1/5, 1×, 2/3, 2×CHEV Diatom Medium; 1:1 DYIII/PEA+Gr+; Ag Diatom Medium; Allen Medium; BG11-1 Medium; Bold 1NV and 3N Medium; Botryococcus Medium; Bristol Medium; Chu's Medium; CR1, CR1−S, and CR1+Diatom Medium; Cyanidium Medium; Cyanophycean Medium; Desmid Medium; DYIII Medium; Euglena Medium; HEPES Medium; J Medium; Malt Medium; MES Medium; Modified Bold 3N Medium; Modified COMBO Medium; N/20 Medium; Ochromonas Medium; P49 Medium; Polytomella Medium; Proteose Medium; Snow Algae Media; Soil Extract Medium; Soilwater: BAR, GR−, GR−/NH4, GR+, GR+/NH4, PEA, Peat, and VT Medium; Spirulina Medium; Tap Medium; Trebouxia Medium; Volvocacean Medium; Volvocacean-3N Medium; Volvox Medium; Volvox-Dextrose Medium; Waris Medium; and Waris+Soil Extract Medium. Various Salt Water Media include: 1%, 5%, and 1×F/2 Medium; 1/2, 1×, and 2× Erdschreiber's Medium; 1/2, 1/3, 1/4, 1/5, 1×, 5/3, and 2× Soil+Seawater Medium; 1/4 ERD; 2/3 Enriched Seawater Medium; 20% Allen+80% ERD; Artificial Seawater Medium; BG11-1+0.36% NaCl Medium; BG11-1+1% NaCl Medium; Bold 1NV:Erdshreiber (1:1) and (4:1); Bristol-NaCl Medium; Dasycladales Seawater Medium; 1/2 and 1× Enriched Seawater Medium, including ES/10, ES/2, and ES/4; F/2+NH4; LDM Medium; Modified 1× and 2×CHEV; Modified 2×CHEV+Soil; Modified Artificial Seawater Medium; Porphridium Medium; and SS Diatom Medium.

Other suitable media for use with the methods of the invention can be readily identified by consulting other organizations that maintain cultures of microorganisms, such as SAG, CCAP, or CCALA. SAG refers to the Culture Collection of Algae at the University of Göttingen (Göttingen, Germany), CCAP refers to the culture collection of algae and protozoa managed by the Scottish Association for Marine Science (Scotland, United Kingdom), and CCALA refers to the culture collection of algal laboratory at the Institute of Botany (T\check{r}ebo\check{n}, Czech Republic).

Microorganisms useful in accordance with the methods of the present disclosure are found in various locations and environments throughout the world. As a consequence of their isolation from other species and their resulting evolutionary divergence, the particular growth medium for optimal growth and generation of oil and/or lipid and/or protein from any particular species of microbe can be difficult or impossible to predict, but those of skill in the art can readily find appropriate media by routine testing in view of the disclosure herein. In some cases, certain strains of microorganisms may be unable to grow on a particular growth medium because of the presence of some inhibitory component or the absence of some essential nutritional requirement required by the particular strain of microorganism. The examples below provide exemplary methods of culturing various species of microalgae to accumulate high levels of lipid as a percentage of dry cell weight.

Suitable fixed carbon sources for use in the medium, include, for example, glucose, fructose, sucrose, galactose, xylose, mannose, rhamnose, arabinose, N-acetylglucosamine, glycerol, floridoside, glucuronic acid, and/or acetate.

High lipid biomass from microalgae is an advantageous material for inclusion in cosmetic products compared to low lipid biomass, because it allows for the addition of less microalgal biomass to incorporate the same amount of lipid into a cosmetic composition. Process conditions can be adjusted to increase the percentage weight of cells that is lipid. For example, in certain embodiments, a microalgae is cultured in the presence of a limiting concentration of one or more nutrients, such as, for example, nitrogen, phosphorous, or sulfur, while providing an excess of a fixed carbon source, such as glucose. Nitrogen limitation tends to increase microbial lipid yield over microbial lipid yield in a culture in which nitrogen is provided in excess. In particular embodiments, the increase in lipid yield is at least about 10%, 50%, 100%, 200%, or 500%. The microbe can be cultured in the presence of a limiting amount of a nutrient for a portion of the total culture period or for the entire period. In some embodiments, the nutrient concentration is cycled between a limiting concentration and a non-limiting concentration at least twice during the total culture period.

In a steady growth state, the cells accumulate oil but do not undergo cell division. In one embodiment of the invention, the growth state is maintained by continuing to provide all components of the original growth media to the cells with the exception of a fixed nitrogen source. Cultivating microalgal cells by feeding all nutrients originally provided to the cells except a fixed nitrogen source, such as through feeding the cells for an extended period of time, results in a higher percentage of lipid by dry cell weight.

In other embodiments, high lipid biomass is generated by feeding a fixed carbon source to the cells after all fixed nitrogen has been consumed for extended periods of time, such as at least one or two weeks. In some embodiments, cells are allowed to accumulate oil in the presence of a fixed carbon source and in the absence of a fixed nitrogen source for over 20 days. Microalgae grown using conditions described herein or otherwise known in the art can comprise at least about 20% lipid by dry weight, and often comprise 35%, 45%, 55%, 65%, and even 75% or more lipid by dry weight. Percentage of dry cell weight as lipid in microbial lipid production can therefore be improved by holding cells in a heterotrophic growth state in which they consume carbon and accumulate oil but do not undergo cell division.

High protein biomass from algae is another advantageous material for inclusion in cosmetic products. The methods of the invention can also provide biomass that has at least 30% of its dry cell weight as protein. Growth conditions can be adjusted to increase the percentage weight of cells that is protein. In a preferred embodiment, a microalgae is cultured in a nitrogen rich environment and an excess of fixed carbon energy such as glucose or any of the other carbon sources discussed above. Conditions in which nitrogen is in excess tends to increase microbial protein yield over microbial protein yield in a culture in which nitrogen is not provided in excess. For maximal protein production, the microbe is preferably cultured in the presence of excess nitrogen for the total culture period. Suitable nitrogen sources for microalgae may come from organic nitrogen sources and/or inorganic nitrogen sources.

Organic nitrogen sources have been used in microbial cultures since the early 1900s. The use of organic nitrogen sources, such as corn steep liquor was popularized with the production of penicillin from mold. Researchers found that the inclusion of corn steep liquor in the culture medium increased the growth of the microorganism and resulted in an increased yield in products (such as penicillin). An analysis of corn steep liquor determined that it was a rich source of nitrogen and also vitamins such as B-complex vitamins, riboflavin panthothenic acid, niacin, inositol and nutrient minerals such as calcium, iron, magnesium, phosphorus and potassium (Ligget and Koffler, Bacteriological Reviews (1948); 12(4): 297-311). Organic nitrogen sources, such as corn steep liquor, have been used in fermentation media for yeasts, bacteria, fungi and other microorganisms. Non-limiting examples of organic nitrogen sources are yeast extract, peptone, corn steep liquor and corn steep powder. Non-limiting examples of preferred inorganic nitrogen sources include, for example, and without limitation, (NH₄)₂SO₄ and NH₄OH. In one embodiment, the culture media for carrying out the invention contains only inorganic nitrogen sources. In another embodiment, the culture media for carrying out the invention contains only organic nitrogen sources. In yet another embodiment, the culture media for carrying out the invention contains a mixture of organic and inorganic nitrogen sources.

In the methods of the invention, a bioreactor or fermentor is used to culture microalgal cells through the various phases of their physiological cycle. As an example, an inoculum of lipid-producing microalgal cells is introduced into the medium; there is a lag period (lag phase) before the cells begin to propagate. Following the lag period, the propagation rate increases steadily and enters the log, or exponential, phase. The exponential phase is in turn followed by a slowing of propagation due to decreases in nutrients such as nitrogen, increases in toxic substances, and quorum sensing mechanisms. After this slowing, propagation stops, and the cells enter a stationary phase or steady growth state, depending on the particular environment provided to the cells. For obtaining protein rich biomass, the culture is typically harvested during or shortly after then end of the exponential phase. For obtaining lipid rich biomass, the culture is typically harvested well after then end of the exponential phase, which may be terminated early by allowing nitrogen or another key nutrient (other than carbon) to become depleted, forcing the cells to convert the carbon sources, present in excess, to lipid. Culture condition parameters can be manipulated to optimize total oil production, the combination of lipid species produced, and/or production of a specific oil.

Bioreactors offer many advantages for use in heterotrophic growth and propagation methods. As will be appreciated, provisions made to make light available to the cells in photosynthetic growth methods are unnecessary when using a fixed-carbon source in the heterotrophic growth and propagation methods described herein. To produce biomass for use in cosmetics, microalgae are preferably fermented in large quantities in liquid, such as in suspension cultures as an example. Bioreactors such as steel fermentors (5000 liter, 10,000 liter, 40,000 liter, and higher are used in various embodiments of the invention) can accommodate very large culture volumes. Bioreactors also typically allow for the control of culture conditions such as temperature, pH, oxygen tension, and carbon dioxide levels. For example, bioreactors are typically configurable, for example, using ports attached to tubing, to allow gaseous components, like oxygen or nitrogen, to be bubbled through a liquid culture.

Bioreactors can be configured to flow culture media though the bioreactor throughout the time period during which the microalgae reproduce and increase in number. In some embodiments, for example, media can be infused into the bioreactor after inoculation but before the cells reach a desired density. In other instances, a bioreactor is filled with culture media at the beginning of a culture, and no more culture media is infused after the culture is inoculated. In other words, the microalgal biomass is cultured in an aqueous medium for a period of time during which the microalgae reproduce and increase in number; however, quantities of aqueous culture medium are not flowed through the bioreactor throughout the time period. Thus in some embodiments, aqueous culture medium is not flowed through the bioreactor after inoculation.

Bioreactors equipped with devices such as spinning blades and impellers, rocking mechanisms, stir bars, means for pressurized gas infusion can be used to subject microalgal cultures to mixing. Mixing may be continuous or intermittent. For example, in some embodiments, a turbulent flow regime of gas entry and media entry is not maintained for reproduction of microalgae until a desired increase in number of said microalgae has been achieved.

As briefly mentioned above, bioreactors are often equipped with various ports that, for example, allow the gas content of the culture of microalgae to be manipulated. To illustrate, part of the volume of a bioreactor can be gas rather than liquid, and the gas inlets of the bioreactor to allow pumping of gases into the bioreactor. Gases that can be beneficially pumped into a bioreactor include air, air/CO₂ mixtures, noble gases, such as argon, and other gases. Bioreactors are typically equipped to enable the user to control the rate of entry of a gas into the bioreactor. As noted above, increasing gas flow into a bioreactor can be used to increase mixing of the culture.

Increased gas flow affects the turbidity of the culture as well. Turbulence can be achieved by placing a gas entry port below the level of the aqueous culture media so that gas entering the bioreactor bubbles to the surface of the culture. One or more gas exit ports allow gas to escape, thereby preventing pressure buildup in the bioreactor. Preferably a gas exit port leads to a “one-way” valve that prevents contaminating microorganisms from entering the bioreactor.

The specific examples of bioreactors, culture conditions, and heterotrophic growth and propagation methods described herein can be combined in any suitable manner to improve efficiencies of microbial growth and lipid and/or protein production.

Concentration of Microalgae after Fermentation

Microalgal cultures generated according to the methods described above yield microalgal biomass in fermentation media. To prepare the biomass for use as a cosmetic composition, the biomass is concentrated, or harvested, from the fermentation medium. At the point of harvesting the microalgal biomass from the fermentation medium, the biomass comprises predominantly intact cells suspended in an aqueous culture medium. To concentrate the biomass, a dewatering step is performed. Dewatering or concentrating refers to the separation of the biomass from fermentation broth or other liquid medium and so is solid-liquid separation. Thus, during dewatering, the culture medium is removed from the biomass (for example, by draining the fermentation broth through a filter that retains the biomass), or the biomass is otherwise removed from the culture medium. Common processes for dewatering include centrifugation, filtration, and the use of mechanical pressure. These processes can be used individually or in any combination.

Centrifugation involves the use of centrifugal force to separate mixtures. During centrifugation, the more dense components of the mixture migrate away from the axis of the centrifuge, while the less dense components of the mixture migrate towards the axis. By increasing the effective gravitational force (i.e., by increasing the centrifugation speed), more dense material, such as solids, separate from the less dense material, such as liquids, and so separate out according to density. Centrifugation of biomass and broth or other aqueous solution forms a concentrated paste comprising the microalgal cells. Centrifugation does not remove significant amounts of intracellular water. In fact, after centrifugation, there may still be a substantial amount of surface or free moisture in the biomass (e.g., upwards of 70%), so centrifugation is not considered to be a drying step.

Filtration can also be used for dewatering. One example of filtration that is suitable for the present invention is tangential flow filtration (TFF), also known as cross-flow filtration. Tangential flow filtration is a separation technique that uses membrane systems and flow force to separate solids from liquids. For an illustrative suitable filtration method, see Geresh, Carb. Polym. 50; 183-189 (2002), which describes the use of a MaxCell A/G Technologies 0.45 uM hollow fiber filter. Also see, for example, Millipore Pellicon® devices, used with 100 kD, 300 kD, 1000 kD (catalog number P2C01MC01), 0.1 uM (catalog number P2VVPPV01), 0.22 uM (catalog number P2GVPPV01), and 0.45 uM membranes (catalog number P2HVMPV01). The retentate preferably does not pass through the filter at a significant level, and the product in the retentate preferably does not adhere to the filter material. TFF can also be performed using hollow fiber filtration systems. Filters with a pore size of at least about 0.1 micrometer, for example about 0.12, 0.14, 0.16, 0.18, 0.2, 0.22, 0.45, or at least about 0.65 micrometers, are suitable. Preferred pore sizes of TFF allow solutes and debris in the fermentation broth to flow through, but not microbial cells.

Dewatering can also be affected with mechanical pressure directly applied to the biomass to separate the liquid fermentation broth from the microbial biomass sufficient to dewater the biomass but not to cause predominant lysis of cells. Mechanical pressure to dewater microbial biomass can be applied using, for example, a belt filter press. A belt filter press is a dewatering device that applies mechanical pressure to a slurry (e.g., microbial biomass taken directly from the fermentor or bioreactor) that is passed between the two tensioned belts through a serpentine of decreasing diameter rolls. The belt filter press can actually be divided into three zones: the gravity zone, where free draining water/liquid is drained by gravity through a porous belt; a wedge zone, where the solids are prepared for pressure application; and a pressure zone, where adjustable pressure is applied to the gravity drained solids.

After concentration, microalgal biomass can be processed, as described hereinbelow, to produce vacuum-packed cake, algal flakes, algal homogenate, algal powder, algal flour, or algal oil.

### Chemical Composition of Microalgal Biomass

The microalgal biomass generated by the culture methods described herein comprises microalgal oil and/or protein as well as other constituents generated by the microorganisms or incorporated by the microorganisms from the culture medium during fermentation.

Microalgal biomass with a high percentage of oil/lipid accumulation by dry weight has been generated using different methods of culture, including methods known in the art. Microalgal biomass with a higher percentage of accumulated oil/lipid is useful in accordance with the present invention. Chlorella vulgaris cultures with up to 56.6% lipid by dry cell weight (DCW) in stationary cultures grown under autotrophic conditions using high iron (Fe) concentrations have been described (Li et al., Bioresource Technology 99(11):4717-22 (2008). Nanochloropsis sp. and Chaetoceros calcitrans cultures with 60% lipid by DCW and 39.8% lipid by DCW, respectively, grown in a photobioreactor under nitrogen starvation conditions have also been described (Rodolfi et al., Biotechnology & Bioengineering (2008)). Parietochloris incise cultures with approximately 30% lipid by DCW when grown phototropically and under low nitrogen conditions have been described (Solovchenko et al., Journal of Applied Phycology 20:245-251 (2008). Chlorella protothecoides can produce up to 55% lipid by DCW when grown under certain heterotrophic conditions with nitrogen starvation (Miao and Wu, Bioresource Technology 97:841-846 (2006)). Other Chlorella species, including Chlorella emersonii, Chlorella sorokiniana and Chlorella minutissima have been described to have accumulated up to 63% oil by DCW when grown in stirred tank bioreactors under low-nitrogen media conditions (Illman et al., Enzyme and Microbial Technology 27:631-635 (2000). Still higher percent lipid by DCW has been reported, including 70% lipid in Dumaliella tertiolecta cultures grown in increased NaCl conditions (Takagi et al., Journal of Bioscience and Bioengineering 101(3): 223-226 (2006)) and 75% lipid in Botryococcus braunii cultures (Banerjee et al., Critical Reviews in Biotechnology 22(3): 245-279 (2002)).

Heterotrophic growth results in relatively low chlorophyll content (as compared to phototrophic systems such as open ponds or closed photobioreactor systems). The reduced chlorophyll content found in heterotrophically grown microalgae (e.g., Chlorella) also reduces the green color in the biomass as compared to phototrophically grown microalgae. Thus, the reduced chlorophyll content avoids an often undesired green coloring associated with cosmetic products containing phototrophically grown microalgae and allows for the incorporation or an increased incorporation of algal biomass into a cosmetic product. In at least one embodiment, the cosmetic product contains heterotrophically grown microalgae of reduced chlorophyll content compared to phototrophically grown microalgae.

Oil rich microalgal biomass generated by the culture methods described herein and useful in accordance with the present invention comprises at least 10% microalgal oil by DCW. In some embodiments, the microalgal biomass comprises at least 15%, 25%, 50%, 75% or at least 90% microalgal oil by DCW.

The microalgal oil of the biomass described herein (or extracted from the biomass) can comprise glycerolipids with one or more distinct fatty acid ester side chains. Glycerolipids are comprised of a glycerol molecule esterified to one, two, or three fatty acid molecules, which can be of varying lengths and have varying degrees of saturation. Specific blends of algal oil can be prepared either within a single species of algae, or by mixing together the biomass (or algal oil) from two or more species of microalgae.

Thus, the oil composition, i.e., the properties and proportions of the fatty acid constituents of the glycerolipids, can also be manipulated by combining biomass (or oil) from at least two distinct species of microalgae. In some embodiments, at least two of the distinct species of microalgae have different glycerolipid profiles. The distinct species of microalgae can be cultured together or separately as described herein, preferably under heterotrophic conditions, to generate the respective oils. Different species of microalgae can contain different percentages of distinct fatty acid constituents in the cell's glycerolipids.

In some embodiments, the microalgal oil is primarily comprised of monounsaturated oil. In some cases, the algal oil is at least 20% monounsaturated oil by weight. In various embodiments, the algal oil is at least 25%, 50%, 75% or more monounsaturated oil by weight or by volume. In some embodiments, the monounsaturated oil is 18:1, 16:1, 14:1 or 12:1. In some embodiments, the microalgal oil comprises at least 10%, 20%, 25%, or 50% or more esterified oleic acid or esterified alpha-linolenic acid by weight of by volume. In at least one embodiment, the algal oil comprises less than 10%, less than 5%, less than 3%, less than 2%, or less than 1% by weight or by volume, or is substantially free of, esterified docosahexanoic acid (DHA (22:6)). For examples of production of high DHA-containing microalgae, such as in Crypthecodinium cohnii, see U.S. Pat. Nos. 7,252,979, 6,812,009 and 6,372,460.

High protein microalgal biomass has been generated using different methods of culture. Microalgal biomass with a higher percentage of protein content is useful in accordance with the present invention. For example, the protein content of various species of microalgae has been reported (see Table 1 of Becker, Biotechnology Advances (2007) 25:207-210). Controlling the renewal rate in a semi-continous photoautotrophic culture of Tetraselmis suecica has been reported to affect the protein content per cell, the highest being approximately 22.8% protein (Fabregas, et al., Marine Biotechnology (2001) 3:256-263).

Microalgal biomass generated by culture methods described herein and useful in accordance to those embodiments of the present invention relating to high protein typically comprises at least 30% protein by dry cell weight. In some embodiments, the microalgal biomass comprises at least 40%, 50%, 75% or more protein by dry cell weight. In some embodiments, the microalgal biomass comprises from 30-75% protein by dry cell weight or from 40-60% protein by dry cell weight. In some embodiments, the protein in the microalgal biomass comprises at least 40% digestible crude protein. In other embodiments, the protein in the microalgal biomass comprises at least 50%, 60%, 70%, 80%, or at least 90% digestible crude protein. In some embodiments, the protein in the microalgal biomass comprises from 40-90% digestible crude protein, from 50-80% digestible crude protein, or from 60-75% digestible crude protein.

Microalgal biomass (and oil extracted therefrom), can also include other constituents produced by the microalgae, or incorporated into the biomass from the culture medium. These other constituents can be present in varying amounts depending on the culture conditions used and the species of microalgae (and, if applicable, the extraction method used to recover microalgal oil from the biomass). The other constituents can include, without limitation, phospholipids (e.g., algal lecithin), carbohydrates, soluble and insoluble fiber, glycoproteins, phytosterols (e.g., β-sitosterol, campesterol, stigmasterol, ergosterol, and brassicasterol), tocopherols, tocotrienols, carotenoids (e.g., α-carotene, β-carotene, and lycopene), xanthophylls (e.g., lutein, zeaxanthin, α-cryptoxanthin, and β-cryptoxanthin), proteins, polysaccharides (e.g., arabinose, mannose, galactose, 6-methyl galactose and glucose) and various organic or inorganic compounds (e.g., selenium). Microalgal sterols may have anti-inflammatory, anti-matrix-breakdown, and improvement of skin barrier effects when incorporated into a skincare product such as described in section IV(f) and Example 26.

In some cases, the biomass comprises at least 10 ppm selenium. In some cases, the biomass comprises at least 25% w/w algal polysaccharide. In some cases, the biomass comprises at least 15% w/w algal glycoprotein. In some cases, the biomass comprises between 0-115 mcg/g total carotenoids. In some cases, the biomass comprises at least 0.5% algal phospholipids. In some cases, the oil derived from the algal biomass contains at least 0.10 mg/g total tocotrienols. In some cases, the oil derived from the algal biomass contains between 0.125 mg/g to 0.35 mg/g total tocotrienols. In some cases, the oil derived from the algal biomass contains at least 5.0 mg/100 g total tocopherols. In some cases, the oil derived from the algal biomass contains between 5.0 mg/100 g to 10 mg/100 g tocopherols. A detailed description of tocotrienols and tocopherols composition in Chlorella protothecoides is included in the Examples below.

Processing Microalgal Biomass into Finished Cosmetic Ingredients

The concentrated microalgal biomass produced in accordance with the methods of the invention is itself a finished cosmetic ingredient and may be used in cosmetics without further, or with only minimal, modification. For example, the cake can be vacuum-packed or frozen. Alternatively, the biomass may be dried via lyophilization, a “freeze-drying” process, in which the biomass is frozen in a freeze-drying chamber to which a vacuum is applied. The application of a vacuum to the freeze-drying chamber results in sublimation (primary drying) and desorption (secondary drying) of the water from the biomass. However, the present invention provides a variety of microalgal derived finished cosmetic ingredients with enhanced properties resulting from processing methods of the invention that can be applied to the concentrated microalgal biomass.

Drying the microalgal biomass, either predominantly intact or in homogenate form, is advantageous to facilitate further processing or for use of the biomass in the methods and compositions described herein. Drying refers to the removal of free or surface moisture/water from predominantly intact biomass or the removal of surface water from a slurry of homogenized (e.g., by micronization) biomass. Different textures and dispersion properties can be conferred on cosmetic products depending on whether the algal biomass is dried, and if so, the drying method. Drying the biomass generated from the cultured microalgae described herein removes water that may be an undesirable component of finished cosmetic products or cosmetic ingredients. In some cases, drying the biomass may facilitate a more efficient microalgal oil extraction process.

In one embodiment, the concentrated microalgal biomass is drum dried to a flake form to produce algal flake, as described in part A of this section. In another embodiment, the concentrated micralgal biomass is spray or flash dried (i.e., subjected to a pneumatic drying process) to form a powder containing predominantly intact cells to produce algal powder, as described in part B of this section. In another embodiment, oil is extracted from the concentrated microalgal biomass to form algal oil, as described in part C of this section.

### A. Algal Flake

Algal flake of the invention is prepared from concentrated microalgal biomass that is applied as a film to the surface of a rolling, heated drum. The dried solids are then scraped off with a knife or blade, resulting in a small flakes. U.S. Pat. No. 6,607,900 describes drying microalgal biomass using a drum dryer without a prior centrifugation (concentration) step, and such a process may be used in accordance with the methods of the invention.

Because the biomass may be exposed to high heat during the drying process, it may be advantageous to add an antioxidant to the biomass prior to drying. The addition of an antioxidant will not only protect the biomass during drying, but also extend the shelf-life of the dried microalgal biomass when stored. In a preferred embodiment, an antioxidant is added to the microalgal biomass prior to subsequent processing such as drying or homogenization. Antioxidants that are suitable for use are discussed in detail below.

Additionally, if there is significant time between the production of the dewatered microalgal biomass and subsequent processing steps, it may be advantageous to pasteurize the biomass prior to drying. Free fatty acids from lipases may form if there is significant time between producing and drying the biomass. In one embodiment, the pasteurized microalgal biomass is an algal flake.

### B. Algal Powder

Algal powder of the invention is prepared from concentrated microalgal biomass using a pneumatic or spray dryer (see for example U.S. Pat. No. 6,372,460). In a spray dryer, material in a liquid suspension is sprayed in a fine droplet dispersion into a current of heated air. The entrained material is rapidly dried and forms a dry powder. In some cases, a pulse combustion dryer can also be used to achieve a powdery texture in the final dried material. In other cases, a combination of spray drying followed by the use of a fluid bed dryer is used to achieve the optimal conditions for dried microbial biomass (see, for example, U.S. Pat. No. 6,255,505). As an alternative, pneumatic dryers can also be used in the production of algal powder. Pneumatic dryers draw or entrain the material that is to be dried in a stream of hot air. While the material is entrained in the hot air, the moisture is rapidly removed. The dried material is then separated from the moist air and the moist air is then recirculated for further drying.

### C. Algal Flour

Algal flour of the invention is prepared from concentrated microalgal biomass that has been mechanically lysed and homogenized and the homogenate spray or flash dried (or dried using another pneumatic drying system). The production of algal flour requires that cells be lysed to release their oil and that cell wall and intracellular components be micronized or reduced in particle size to an average size of no more than 10 μm. The resulting oil, water, and micronized particles are emulsified such that the oil does not separate from the dispersion prior to drying. For example, a pressure disrupter can be used to pump a cell containing slurry through a restricted orifice valve to lyse the cells. High pressure (up to 1500 bar) is applied, followed by an instant expansion through an exiting nozzle. Cell disruption is accomplished by three different mechanisms: impingement on the valve, high liquid shear in the orifice, and sudden pressure drop upon discharge, causing an explosion of the cell. The method releases intracellular molecules. A Niro (Niro Soavi GEA) homogenizer (or any other high pressure homogenizer) can be used to process cells to particles predominantly 0.2 to 5 microns in length. Processing of algal biomass under high pressure (approximately 1000 bar) typically lyses over 90% of the cells and reduces particle size to less than 5 microns.

Alternatively, a ball mill can be used. In a ball mill, cells are agitated in suspension with small abrasive particles, such as beads. Cells break because of shear forces, grinding between beads, and collisions with beads. The beads disrupt the cells to release cellular contents. In one embodiment, algal biomass is disrupted and formed into a stable emulsion using a Dyno-mill ECM Ultra (CB Mills) ball mill. Cells can also be disrupted by shear forces, such as with the use of blending (such as with a high speed or Waring blender as examples), the french press, or even centrifugation in case of weak cell walls, to disrupt cells. A suitable ball mill including specifics of ball size and blade is described in U.S. Pat. No. 5,330,913.

The immediate product of homogenization is a slurry of particles smaller in size than the original cells that is suspended in oil and water. The particles represent cellular debris. The oil and water are released by the cells. Additional water may be contributed by aqueous media containing the cells before homogenization. The particles are preferably in the form of a micronized homogenate. If left to stand, some of the smaller particles may coalesce. However, an even dispersion of small particles can be preserved by seeding with a microcrystalline stabilizer, such as microcrystalline cellulose.

To form the algal flour, the slurry is spray or flash dried, removing water and leaving a dry power containing cellular debris and oil. Although the oil content of the powder can be at least 10, 25 or 50% by weight of the dry powder, the powder can have a dry rather than greasy feel and appearance (e.g., lacking visible oil) and can also flow freely when shaken. Various flow agents (including silica-derived products) can also be added. After drying, the water or moisture content of the powder is typically less than 10%, 5%, 3% or 1% by weight. Other dryers such as pneumatic dryers or pulse combustion dryers can also be used to produce algal flour.

The oil content of algal flour can vary depending on the percent oil of the algal biomass. Algal flour can be produced from algal biomass of varying oil content. In certain embodiments, the algal flour is produced from algal biomass of the same oil content. In other embodiments, the algal flour is produced from alglal biomass of different oil content. In the latter case, algal biomass of varying oil content can be combined and then the homogenization step performed. In other embodiments, algal flour of varying oil content is produced first and then blended together in various proportions in order to achieve an algal flour product that contains the final desired oil content. In a further embodiment, algal biomass of different lipid profiles can be combined together and then homogenized to produce algal flour. In another embodiment, algal flour of different lipid profiles is produced first and then blended together in various proportions in order to achieve an algal flour product that contains the final desired lipid profile.

### D. Algal Oil

In one aspect, the present invention is directed to a method of preparing algal oil by harvesting algal oil from an algal biomass comprising at least 15% oil by dry weight under GMP conditions, in which the algal oil is greater than 50% 18:1 lipid. In some cases, the algal biomass comprises a mixture of at least two distinct species of microalgae. In some cases, at least two of the distinct species of microalgae have been separately cultured. In at least one embodiment, at least two of the distinct species of microalgae have different glycerolipid profiles. In some cases, the algal biomass is derived from algae grown heterotrophically. In some cases, all of the at least two distinct species of microalgae contain at least 15% oil by dry weight.

In one aspect, the present invention is directed to a method of making a cosmetic composition comprising combining algal oil obtained from algal cells containing at least 10%, or at least 15% oil by dry weight with one or more other ingredients to form the cosmetic composition. In some cases, the method further comprises preparing the algal oil under GMP conditions.

Algal oil can be separated from lysed biomass for use in cosmetic products (among other applications). The algal biomass remaining after oil extraction is referred to as delipidated meal. Delipidated meal contains less oil by dry weight or volume than the microalgae contained before extraction. Typically 50-90% of oil is extracted so that delipidated meal contains, for example, 10-50% of the oil content of biomass before extraction. However, the biomass still has a high nutrient value in content of protein and other constituents discussed above. Thus, the delipidated meal can be used in animal feed or in human food applications.

In some embodiments, the algal oil is at least 50% w/w oleic acid and contains less than 5% DHA. In some embodiments of the method, the algal oil is at least 50% w/w oleic acid and contains less than 0.5% DHA. In some embodiments of the method, the algal oil is at least 50% w/w oleic acid and contains less than 5% glycerolipid containing carbon chain length greater than 18. In some cases, the algal cells from which the algal oil is obtained comprise a mixture of cells from at least two distinct species of microalgae. In some cases, at least two of the distinct species of microalgae have been separately cultured. In at least one embodiment, at least two of the distinct species of microalgae have different glycerolipid profiles. In some cases, the algal cells are cultured under heterotrophic conditions. In some cases, all of the at least two distinct species of microalgae contain at least 10%, or at least 15% oil by dry weight.

In one aspect, provided is an algal oil containing at least 50% monounsaturated oil and containing less than 1% DHA prepared under GMP conditions. In some cases, the monounsaturated oil is 18:1 lipid. In some cases, the algal oil is packaged in a capsule for delivery of a unit dose of oil. In some cases, the algal oil is derived from a mixture of at least two distinct species of microalgae. In some cases, at least two of the distinct species of microalgae have been separately cultured. In at least one embodiment, at least two of the distinct species of microalgae have different glycerolipid profiles. In some cases, the algal oil is derived from algal cells cultured under heterotrophic conditions.

In one aspect, provided is an oil comprising greater than 60% 18:1, and at least 0.20 mg/g tocotrienol.

In one aspect, provided is a fatty acid alkyl ester composition comprising greater than 60% 18:1 ester, and at least 0.20 mg/g tocotrienol.

In one aspect, the algal oil is prepared from concentrated, washed microalgal biomass by extraction. The cells in the biomass are lysed prior to extraction. Optionally, the microbial biomass may also be dried (oven dried, lyophilized, etc.) prior to lysis (cell disruption). Alternatively, cells can be lysed without separation from some or all of the fermentation broth when the fermentation is complete. For example, the cells can be at a ratio of less than 1:1 v:v cells to extracellular liquid when the cells are lysed.

Microalgae containing lipids can be lysed to produce a lysate. As detailed herein, the step of lysing a microorganism (also referred to as cell lysis) can be achieved by any convenient means, including heat-induced lysis, adding a base, adding an acid, using enzymes such as proteases and polysaccharide degradation enzymes such as amylases, using ultrasound, mechanical pressure-based lysis, and lysis using osmotic shock. Each of these methods for lysing a microorganism can be used as a single method or in combination simultaneously or sequentially. The extent of cell disruption can be observed by microscopic analysis. Using one or more of the methods above, typically more than 70% cell breakage is observed. Preferably, cell breakage is more than 80%, more preferably more than 90% and most preferred about 100%.

Lipids and oils generated by the microalgae in accordance with the present invention can be recovered by extraction. In some cases, extraction can be performed using an organic solvent or an oil, or can be performed using a solventless-extraction procedure.

For organic solvent extraction of the microalgal oil, the preferred organic solvent is hexane. Typically, the organic solvent is added directly to the lysate without prior separation of the lysate components. In one embodiment, the lysate generated by one or more of the methods described above is contacted with an organic solvent for a period of time sufficient to allow the lipid components to form a solution with the organic solvent. In some cases, the solution can then be further refined to recover specific desired lipid components. The mixture can then be filtered and the hexane removed by, for example, rotoevaporation. Hexane extraction methods are well known in the art. See, e.g., Frenz et al., Enzyme Microb. Technol., 11:717 (1989).

Miao and Wu describe a protocol of the recovery of microalgal lipid from a culture of Chlorella protothecoides in which the cells were harvested by centrifugation, washed with distilled water and dried by freeze drying. The resulting cell powder was pulverized in a mortar and then extracted with n-hexane. Miao and Wu, Biosource Technology 97:841-846 (2006).

In some cases, microalgal oils can be extracted using liquefaction (see for example Sawayama et al., Biomass and Bioenergy 17:33-39 (1999) and Inoue et al., Biomass Bioenergy 6(4):269-274 (1993)); oil liquefaction (see for example Minowa et al., Fuel 74(12):1735-1738 (1995)); or supercritical CO₂ extraction (see for example Mendes et al., Inorganica Chimica Acta 356:328-334 (2003)).

Oil extraction includes the addition of an oil directly to a lysate without prior separation of the lysate components. After addition of the oil, the lysate separates either of its own accord or as a result of centrifugation or the like into different layers. The layers can include in order of decreasing density: a pellet of heavy solids, an aqueous phase, an emulsion phase, and an oil phase. The emulsion phase is an emulsion of lipids and aqueous phase. Depending on the percentage of oil added with respect to the lysate (w/w or v/v), the force of centrifugation if any, volume of aqueous media and other factors, either or both of the emulsion and oil phases can be present. Incubation or treatment of the cell lysate or the emulsion phase with the oil is performed for a time sufficient to allow the lipid produced by the microorganism to become solubilized in the oil to form a heterogeneous mixture.

In various embodiments, the oil used in the extraction process is selected from the group consisting of oil from soy, rapeseed, canola, palm, palm kernel, coconut, corn, waste vegetable oil, Chinese tallow, olive, sunflower, cotton seed, chicken fat, beef tallow, porcine tallow, microalgae, macroalgae, Cuphea, flax, peanut, choice white grease (lard), Camelina sativa mustard seedcashew nut, oats, lupine, kenaf, calendula, hemp, coffee, linseed, hazelnut, euphorbia, pumpkin seed, coriander, camellia, sesame, safflower, rice, tung oil tree, cocoa, copra, pium poppy, castor beans, pecan, jojoba, jatropha, macadamia, Brazil nuts, and avocado. The amount of oil added to the lysate is typically greater than 5% (measured by v/v and/or w/w) of the lysate with which the oil is being combined. Thus, a preferred v/v or w/w of the oil is greater than 5%, 10%, 20%, 25%, 50%, 70%, 90%, or at least 95% of the cell lysate.

Lipids can also be extracted from a lysate via a solventless extraction procedure without substantial or any use of organic solvents or oils by cooling the lysate. Sonication can also be used, particularly if the temperature is between room temperature and 65° C. Such a lysate on centrifugation or settling can be separated into layers, one of which is an aqueous:lipid layer. Other layers can include a solid pellet, an aqueous layer, and a lipid layer. Lipid can be extracted from the emulsion layer by freeze thawing or otherwise cooling the emulsion. In such methods, it is not necessary to add any organic solvent or oil. If any solvent or oil is added, it can be below 5% v/v or w/w of the lysate.

The oils produced according to the above methods in some cases are made using a microalgal host cell. As described above, the microalga can be, without limitation, fall in the classification of Chlorophyta, Trebouxiophyceae, Chlorellales, Chlorellaceae, or Chlorophyceae. It has been found that microalgae of Trebouxiophyceae can be distinguished from vegetable oils based on their sterol profiles. Oil produced by Chlorella protothecoides was found to produce sterols that appeared to be brassicasterol, ergosterol, campesterol, stigmasterol, and β-sitosterol, when detected by GC-MS. However, it is believed that all sterols produced by Chlorella have C24β stereochemistry. Thus, it is believed that the molecules detected as campesterol, stigmasterol, and β-sitosterol, are actually 22,23-dihydrobrassicasterol, proferasterol and clionasterol, respectively. Thus, the oils produced by the microalgae described above can be distinguished from plant oils by the presence of sterols with C24β stereochemistry and the absence of C24α stereochemistry in the sterols present. For example, the oils produced may contain 22,23-dihydrobrassicasterol while lacking campesterol; contain clionasterol, while lacking in β-sitosterol, and/or contain poriferasterol while lacking stigmasterol. Alternately, or in addition, the oils may contain significant amounts of Δ⁷-poriferasterol.

In one embodiment, the oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

Sterol profile analysis is a particularly well-known method for determining the biological source of organic matter. Campesterol, b-sitosterol, and stigamsterol are common plant sterols, with b-sitosterol being a principle plant sterol. For example, b-sitosterol was found to be in greatest abundance in an analysis of certain seed oils, approximately 64% in corn, 29% in rapeseed, 64% in sunflower, 74% in cottonseed, 26% in soybean, and 79% in olive oil (Gul et al. J. Cell and Molecular Biology 5:71-79, 2006).

Oil isolated from Prototheca moriformis strain UTEX1435 were separately clarified (CL), refined and bleached (RB), or refined, bleached and deodorized (RBD) and were tested for sterol content according to the procedure described in JAOCS vol. 60, no. 8, August 1983. Results of the analysis are shown below (units in mg/100 g):

| | | | | | Refined, |
|---|---|---|---|---|---|
| | | | | Refined & | bleached, & |
| | Sterol | Crude | Clarified | bleached | deodorized |
| 1 | Ergosterol | 384 | 398 | 293 | 302 |
| | | (56%) | (55%) | (50%) | (50%) |
| 2 | 5,22-cholestadien- | 14.6 | 18.8 | 14 | 15.2 |
| | 24-methyl-3-ol | (2.1%) | (2.6%) | (2.4%) | (2.5%) |
| | (Brassicasterol) | | | | |
| 3 | 24-methylcholest-5- | 10.7 | 11.9 | 10.9 | 10.8 |
| | en-3-ol | (1.6%) | (1.6%) | (1.8%) | (1.8%) |
| | (Campersterol | | | | |
| | or 22,23- | | | | |
| | dihydrobrassica- | | | | |
| | sterol) | | | | |
| 4 | 5,22-cholestadien- | 57.7 | 59.2 | 46.8 | 49.9 |
| | 24-ethyl-3-ol | (8.4%) | (8.2%) | (7.9%) | (8.3%) |
| | (Stigmaserol or | | | | |
| | poriferasterol) | | | | |
| 5 | 24-ethylcholest-5- | 9.64 | 9.92 | 9.26 | 10.2 |
| | en-3-ol (β-Sitosterol | (1.4%) | (1.4%) | (1.6%) | (1.7%) |
| | or clionasterol) | | | | |
| 6 | Other sterols | 209 | 221 | 216 | 213 |
| | Total sterols | 685.64 | 718.82 | 589.96 | 601.1 |

These results show three striking features. First, ergosterol was found to be the most abundant of all the sterols, accounting for about 50% or more of the total sterols. The amount of ergosterol is greater than that of campesterol, β-sitosterol, and stigamsterol combined. Ergosterol is steroid commonly found in fungus and not commonly found in plants, and its presence particularly in significant amounts serves as a useful marker for non-plant oils. Secondly, the oil was found to contain brassicasterol. With the exception of rapeseed oil, brassicasterol is not commonly found in plant based oils. Thirdly, less than 2% β-sitosterol was found to be present. β-sitosterol is a prominent plant sterol not commonly found in microalgae, and its presence particularly in significant amounts serves as a useful marker for oils of plant origin. In summary, Prototheca moriformis strain UTEX1435 has been found to contain both significant amounts of ergosterol and only trace amounts of β-sitosterol as a percentage of total sterol content. Accordingly, the ratio of ergosterol:β-sitosterol or in combination with the presence of brassicasterol can be used to distinguish this oil from plant oils.

In some embodiments, the oil content of an oil provided herein contains, as a percentage of total sterols, less than 20%, 15%, 10%, 5%, 4%, 3%, 2%, or 1% β-sitosterol. In other embodiments the oil is free from β-sitosterol.

In some embodiments, the oil is free from one or more of β-sitosterol, campesterol, or stigmasterol. In some embodiments the oil is free from β-sitosterol, campesterol, and stigmasterol. In some embodiments the oil is free from campesterol. In some embodiments the oil is free from stigmasterol.

In some embodiments, the oil content of an oil provided herein comprises, as a percentage of total sterols, less than 20%, 15%, 10%, 5%, 4%, 3%, 2%, or 1% 24-ethylcholest-5-en-3-ol. In some embodiments, the 24-ethylcholest-5-en-3-ol is clionasterol. In some embodiments, the oil content of an oil provided herein comprises, as a percentage of total sterols, at least 1%, 2%, 3%, 4%, 5%, 6%, 7%, 8%, 9%, or 10% clionasterol.

In some embodiments, the oil content of an oil provided herein contains, as a percentage of total sterols, less than 20%, 15%, 10%, 5%, 4%, 3%, 2%, or 1% 24-methylcholest-5-en-3-ol. In some embodiments, the 24-methylcholest-5-en-3-ol is 22,23-dihydrobrassicasterol. In some embodiments, the oil content of an oil provided herein comprises, as a percentage of total sterols, at least 1%, 2%, 3%, 4%, 5%, 6%, 7%, 8%, 9%, or 10% 22,23-dihydrobrassicasterol.

In some embodiments, the oil content of an oil provided herein contains, as a percentage of total sterols, less than 20%, 15%, 10%, 5%, 4%, 3%, 2%, or 1% 5,22-cholestadien-24-ethyl-3-ol. In some embodiments, the 5,22-cholestadien-24-ethyl-3-ol is poriferasterol. In some embodiments, the oil content of an oil provided herein comprises, as a percentage of total sterols, at least 1%, 2%, 3%, 4%, 5%, 6%, 7%, 8%, 9%, or 10% poriferasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4% or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

In some embodiments, the oil content contains, as a percentage of total sterols, at least 5%, 10%, 20%, 25%, 35%, 40%, 45%, 50%, 55%, 60%, or 65% ergosterol and less than 20%, 15%, 10%, 5%, 4%, 3%, 2%, or 1% β-sitosterol. In some embodiments, the oil content contains, as a percentage of total sterols, at least 25% ergosterol and less than 5% β-sitosterol. In some embodiments, the oil content further comprises brassicasterol.

Combining Microalgal Biomass or Materials Derived Therefrom with Other Cosmetic Ingredients

In one aspect, the present invention is directed to methods of combining microalgal biomass and/or microalgal oil, as described above, with at least one other cosmetic ingredient, as described below, to form a cosmetic composition.

In some cases, the cosmetic composition formed by the combination of microalgal biomass and/or microalgal oil comprises at least 1%, at least 5%, at least 10%, at least 25%, or at least 50% w/w microalgal biomass or microalgal oil, respectively. In some embodiments, cosmetic compositions formed as described herein comprise at least 2%, at least 3%, at least 4%, at least 15%, at least 20%, at least 30%, at least 35%, at least 40%, at least 45%, at least 55%, at least 60%, at least 65%, at least 70%, at least 75%, at least 80%, at least 85%, at least 90%, or at least 95% w/w microalgal biomass or microalgal oil.

In some cases, the cosmetic composition comprises predominantly intact microalgal cells. In some cases, the cosmetic composition comprises at least 50% intact cells, or at least 60%, at least 70%, or at least 80% intact cells. In other embodiments, the cosmetic composition comprises microalgal biomass that has been homogenized to form a whole cell dispersion.

### A. Substitution of Algal Biomass and Algal Oil in Cosmetic Products

In some cases, microalgal biomass can be substituted for other components that would otherwise be conventionally included in a cosmetic product. In at least one embodiment, the cosmetic composition formed by the methods of the invention is free of oil other than microalgal oil contributed by the microalgal biomass and entrapped therein.

In various embodiments, microalgal biomass can be substituted for all or a portion of conventional cosmetic ingredients such as exfoliants, antioxidants, colorants, and the like, to the extent that the components of the microalgal biomass replace the corresponding conventional components in like kind, or adequately substitute for the conventional components to impart the desired characteristics to the cosmetic composition.

In some cases, microalgal oil can be substituted for oils conventionally used in cosmetic compositions. As described herein, oils produced by microalgae can be tailored by culture conditions or lipid pathway engineering to comprise particular fatty acid components. Thus, the oils generated by the microalgae of the present invention can be used to replace conventional cosmetic ingredients such as essential oils, fragrance oils, and the like. In at least one embodiment, the cosmetic composition formed by the methods of the present invention is free of oil other than microalgal oil.

### B. Other Cosmetic Ingredients

Microalgal biomass and microalgal oil are combined with at least one other cosmetic ingredients in methods of the present invention to form cosmetic compositions. The at least one other cosmetic ingredient can be selected from conventional cosmetic ingredients suitable for use with the microalgal biomass or microalgal oil with regard to the intended use of the composition. Such other cosmetic ingredients include, without limitation, absorbents, abrasives, anticaking agents, antifoaming agents, antibacterial agents, binders, biological additives, buffering agents, bulking agents, chemical additives, cosmetic biocides, denaturants, cosmetic astringents, drug astringents, external analgesics, film formers, humectants, opacifying agents, fragrances and flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts.

Microalgal biomass and microalgal oil can also be combined with polysaccharides, including polysaccharides from microalgae. Examples of such polysaccharides can be found, for example, in PCT/US2007/001653 “Microalgae-derived Compositions for Improving the Health and Appearance of Skin”, including beads of partially soluble polysaccharides.

Essential oils include allspice, amyris, angelica root, anise seed, basil, bay, bergamot, black pepper, cajeput, camphor, cananga, cardamom, carrot seed, cassia, catnip, cedarwood, chamomile, cinnamon bark, cinnamon leaf, citronella java, clary sage, clovebud, coriander, cornmint, cypress, davana, dill seed, elemi, eucalyptus, fennel, fir, frankincense, geranium bourbon, geranium roast, geranium, ginger, grapefruit pink, grapefruit, gurjum balsam, hyssop, juniper berry, lavandin, lavandula, lavender, lemon myrtle, lemon tea tree, lemon, lemongrass, lime, litsea cubeba, mandarin, marjoram, mullein, myrrh, neroli, nerolina, niaouli, nutmeg, orange, palmarosa, patchouli, peppermint, petitgrain, pine needle, ravensara, ravintsara, rosalina, rose, rosemary, rosewood, sage, sandalwood, spearmint, spikenard, star anise, tangerine, tea tree, thyme, tulsi, verbena, vetiver, ylang ylang, and zdravetz, or combinations thereof.

Fragrances and flavor oils include absolute tulip, almond, amaretto, amber, anais, apple, apple cinnamon, apple spice, apricot, apricot crème, arabian musk, asian pear, asian plum blossom, autumn woods, banana, basil, basil nectarine, bay rum, bayberry, bergamot, berries and cream, birthday cake, black cherry, black tea, blackberry tea, blackcurrent, blue nile, blueberry delight, brambleberry preserves, brown sugar, bubble gum, buttercream, butterscotch, calla lily, cantaloupe, caramel apple, carnation, carrot cake, chai tea, chamomile, china musk, china rain, chinese peony, chrysanthemum, cinnamon, coconut, coconut cream, cotton candy, cranberry, cucumber, cucumber melon, daffodil, dandelion, delphinium, dewberry, dulce de leche, earl grey tea, easter cookie, egg nog, eqyptian musk, enchanted forest, english lavender, english pear, evergreen, fig, frangipani, frankincense, french vanilla, fresh apple, fresh brewed coffee, fruit punch, gardenia, geranium, ginger lily, gingerbread, grape, grapefruit, green apple, green grass, green tea, guava, guava flower, hawaiian white ginger, heliotrope, hemp, herbaceous, holiday fruitcake, hollyberry, honey ginger, honey, honeysuckle, jasmine, jasmine tea, juniper berries, kiwi, lavender, leather, lemon, lemon parsley, lilac, lime, loganberry, lotus blossom, magnolia, mandarin, mango, mango and kiwi, maple, milk chocolate, mimosa, minty lime, mulberry, myrrh, neroli, oakmoss, oatmeal, ocean rain, orange blossom, orange sherbet, orange vanilla, papaya, passion fruit, patchouli, peach, peaches and cream, pearberry, peppermint, pikaki, pina colada, pineapple, pomegranate, pumpkin pie, raisins and almonds, raspberry, roasted nuts, rosewood, sage, sandalwood, sassafras, sea moss, sesame, siberian pine, snowberry, spanish moss, spice, strawberry, sugar plum, suntan lotion, sweet clove, sweet grass, sweet pea, tangerine, thai coconut, timber, tomato leaf, vanilla, watermelon, white chocolate, wild cherry, wisteria, witches brew, and ylang ylang, or combinations thereof.

Exfoliants include particles that can be used to dislodge dead skin cells, dirt, or other materials from the surface of the skin, and include without limitation, fruit seeds and fibers, grain powders, nut and seed meals, and oil or wax beads. Fruit fibers include blueberry, cranberry, grape, kiwi, raspberry, blackberry, strawberry, and the like. Grain powders include oat powder, and almond powder, or the like, milled to varying degrees of coarseness. Polymer beads, such as those made from polyethylene, or the like, can also be used. The removal of dead skin cells and/or the outer most layer of skin can provide an opportunity for bioactive agents, such as carotenoids, which can also be present in the compositions of the invention, to have greater access to deeper layers of the skin.

Extracts, including CO₂ extracts, include herbal extracts derived from conventional extraction procedure, or via the use of liquefied carbon dioxide. Herbs include aloe vera leaf, alfalfa leaf, alkanet root, annatto seed, arrowroot, burdock root, calendula petals, carrot root, chamomile flower, comfrey leaf, cornsilk, dutch blue poppies, fennel seed, ginger root, ginseng, green tea leaf, jasmine flower, juniper berries, lavender buds, lemon peel, lemongrass, marshmallow root, nettles, oat straw, orange peel, paprika, parsley, peppermint leaf, rose buds, rose petals, rosehip, rosemary leaf, shavegrass, spearmint leaf, and st. john's wort, or combinations thereof.

Colorings, including glitters, include green #5, green #8, orange #4, red #22, red #33, violet #2, blue #1, green #3, red #40, yellow #5, yellow #6, green #6, red #17, as well as pearlescent micas and tinting herbs such as henna leaf, sandalwood, turmeric, cranberry, kiwi, raspberry, alkanet, annatto, carrot root, nettles, paprika, and parsley.

Specific examples of other cosmetic ingredients are described below. Any one or more of these can be optionally combined with microalgal biomass or microalgal oil in accordance with the present invention to form a cosmetic composition. The active ingredients described below are categorized by their cosmetic and/or therapeutic benefit or their postulated mode of action. However, it is to be understood that these ingredients can in some instances provide more than one cosmetic and/or therapeutic benefit or operate via more than one mode of action. Therefore, classifications herein are made for the sake of convenience and are not intended to limit the ingredient to that particular application or applications listed.

A safe and effective amount of an anti-inflammatory agent can optionally be added to the compositions of the present invention, preferably from about 0.1% to about 10%, more preferably from about 0.5% to about 5%, of the composition. The anti-inflammatory agent enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

A second class of anti-inflammatory agents which is useful in the compositions includes the nonsteroidal anti-inflammatory agents. The variety of compounds encompassed by this group are well-known to those skilled in the art. For detailed disclosure of the chemical structure, synthesis, side effects, etc. of nonsteroidal anti-inflammatory agents, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974), each incorporated herein by reference.

Specific non-steroidal anti-inflammatory agents useful in accordance with the present invention include, but are not limited to: 1) the oxicams, such as piroxicam, isoxicam, tenoxicam, sudoxicam, and CP-14,304; 2) the salicylates, such as aspirin, disalcid, benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is particularly useful for topical application. Of the nonsteroidal anti-inflammatory agents, ibuprofen, naproxen, flufenamic acid, etofenamate, aspirin, mefenamic acid, meclofenamic acid, piroxicam and felbinac are preferred; ibuprofen, naproxen, etofenamate, aspirin and flufenamic acid are most preferred.

Finally, so-called “natural” anti-inflammatory agents are useful in methods of the present invention. Such agents may suitably be obtained as an extract by suitable physical and/or chemical isolation from natural sources (e.g., plants, fungi, or by-products of microorganisms). For example, candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

In some embodiments, the compositions of the present invention also optionally contain a retinoid. The vitamin B₃ compound and retinoid provide unexpected benefits in regulating skin condition, especially in therapeutically regulating signs of skin aging, more especially wrinkles, lines, and pores. Without intending to be bound or otherwise limited by theory, it is believed that the vitamin B₃ compound increases the conversion of certain retinoids to trans-retinoic acid, which is believed to be the biologically active form of the retinoid, to provide synergistic regulation of skin condition (namely, increased conversion for retinol, retinol esters, and retinal). In addition, the vitamin B₃ compound unexpectedly mitigates redness, inflammation, dermatitis and the like which may otherwise be associated with topical application of retinoid (often referred to, and hereinafter alternatively referred to as “retinoid dermatitis”). Furthermore, the combined vitamin B₃ compound and retinoid tend to increase the amount and activity of thioredoxin, which tends to increase collagen expression levels via the protein AP-1. Therefore, compositions of the present invention enable reduced active levels, and therefore reduced potential for retinoid dermatitis, while retaining significant positive skin conditioning benefits. In addition, higher levels of retinoid may still be used to obtain greater skin conditioning efficacy, without undesirable retinoid dermatitis occurring.

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably retinol, retinol esters (e.g., C₂-C₂₂ alkyl esters of retinol, including retinyl palmitate, retinyl acetate, retinyl proprionate), retinal, and/or retinoic acid (including all-trans retinoic acid and/or 13-cis-retinoic acid), more preferably retinoids other than retinoic acid. These compounds are well known in the art and are commercially available from a number of sources, e.g., Sigma Chemical Company (St. Louis, Mo.).

The cosmetic compositions of this invention may contain a safe and effective amount of the retinoid, such that the resultant composition is safe and effective for regulating skin condition, preferably for regulating visible and/or tactile discontinuities in skin, more preferably for regulating signs of skin aging, even more preferably for regulating visible and/or tactile discontinuities in skin texture associated with skin aging. The compositions preferably contain from or about 0.005% to or about 2%, more preferably 0.01% to or about 2%, retinoid. Retinol is most preferably used in an amount of from or about 0.01% to or about 0.15%; retinol esters (e.g., retinyl acetate or retinyl palmitate) are most preferably used in an amount of from or about 0.01% to or about 2% (e.g., about 1%); retinoic acids are most preferably used in an amount of from or about 0.01% to or about 0.25%. The retinoid may be included as the substantially pure material, or as an extract obtained by suitable physical and/or chemical isolation from natural (e.g., plant) sources. The retinoid is preferably substantially pure.

In some embodiments, the compositions of the present invention also optionally contain an antibacterial agent. As used herein, “antibacterial agent” means a compound capable of destroying bacteria cells, preventing the development of bacteria or preventing the pathogenic action of bacteria. Antibacterial agents are useful, for example, in controlling acne. A safe and effective amount of an antibacterial agent can optionally be added to cosmetic compositions of the subject invention, preferably from about 0.001% to about 10%, more preferably from about 0.01% to about 5%, also from about 0.05% to about 2% or from about 0.05% to about 1% of the compositions. Preferred antibacterial agents useful in the cosmetic compositions of the invention are benzoyl peroxide, erythromycin, tetracycline, clindamycin, azelaic acid, and sulfur resorcinol.

In some embodiments, the compositions of the present invention also optionally contain an antiandrogen. As used herein, “anti-androgen” means a compound capable of correcting androgen-related disorders by interfering with the action of androgens at their target organs. The target organ for the cosmetic compositions of the present invention is mammalian skin. Exemplary antiandrogens include pregnenalone (and its derivatives), hops extract, oxygenated alkyl substituted bicyclo alkanes (e.g., ethoxyhexyl-bicyclo octanones such as marketed by Chantal Pharmaceutical of Los Angeles, Calif. under the trade names ETHOCYN and CYOCTOL, and 2-(5-ethoxy hept-1-yl)bicylo[3.3.0]octanone), and oleanolic acid. Suitable antiandrogens are disclosed in U.S. Pat. Nos. 4,689,345 and 4,855,322, both issued to Kasha et al. on Aug. 25, 1987 and Aug. 8, 1989, respectively, each incorporated herein by reference. Antiandrogens can optionally be added to cosmetic compositions of the invention.

Exposure to ultraviolet light can result in excessive scaling and texture changes of the stratum corneum. Therefore, the cosmetic compositions of the subject invention optionally contain a sunscreen or sunblock. Suitable sunscreens or sunblocks may be organic or inorganic.

A wide variety of conventional sunscreening agents are suitable for use in the cosmetic compositions described herein. Sagarin, et al., at Chapter VIII, pages 189 et seq., of Cosmetics Science and Technology (1972), discloses numerous suitable agents, and is incorporated herein by reference. Specific suitable sunscreening agents include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one) and 4-isopropyl-di-benzoylmethane.

Also optionally useful in the cosmetic compositions are sunscreens such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991, both of which are incorporated herein by reference. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. Members of this class of sunscreening agents include 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxyl)benzophenone; 4-N,N-(2-ethylhexyl)-methylaminobenzoic acid ester of 4-(2-hydroxyethoxyl)dibenzoylmethane; N,N-di-(2-ethylhexyl)-4-aminobenzoic acid ester of 2-hydroxy-4-(2-hydroxyethoxyl)benzophenone; and N,N-di-(2-ethylhexyl)-4-aminobenzoic acid ester of 4-(2-hydroxyethoxyl)dibenzoylmethane and mixtures thereof.

Suitable inorganic sunscreens or sunblocks include metal oxides, e.g., zinc oxide and titanium dioxide.

A safe and effective amount of the sunscreen or sunblock is used, typically from about 1% to about 20%, more typically from about 2% to about 10%. Exact amounts will vary depending upon the sunscreen chosen and the desired Sun Protection Factor (SPF).

An agent may also be added to any of the compositions useful in the subject invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, Brock, issued May 5, 1987, which is incorporated herein by reference.

Cosmetic compositions of the present invention can optionally include an anti-oxidant/radical scavenger as an active ingredient. The anti-oxidant/radical scavenger is especially useful for providing protection against UV radiation which can cause increased scaling or texture changes in the stratum corneum and against other environmental agents which can cause skin damage.

A safe and effective amount of an anti-oxidant/radical scavenger may be added to the compositions of the subject invention, preferably from about 0.1% to about 10%, more preferably from about 1% to about 5%, of the composition.

Anti-oxidants/radical scavengers such as ascorbic acid (vitamin C) and its salts, ascorbyl esters of fatty acids, ascorbic acid derivatives (e.g., magnesium ascorbyl phosphate), tocopherol (vitamin E), tocopherol sorbate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, amines (e.g., N,N-diethylhydroxylamine, amino-guanidine), sulfhydryl compounds (e.g., glutathione), dihydroxy fumaric acid and its salts, lycine pidolate, arginine pilolate, nordibydroguaiaretic acid, bioflavonoids, lysine, methionine, proline, catalase, superoxide dismutase, lactoferrin, silymarin, tea extracts, grape skin/seed extracts, melanin, and rosemary extracts may be used.

As used herein, “chelating agent” refers to an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin texture changes and against other environmental agents which can cause skin damage.

A safe and effective amount of a chelating agent can optionally be added to the cosmetic compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995; all incorporated herein by reference. Preferred chelators useful in compositions of the subject invention are furildioxime and derivatives thereof.

Compositions of the present invention optionally comprise an organic hydroxy acid. Suitable hydroxy acids include C₁-C₁₈ hydroxy acids, preferably C₈ or below. The hydroxy acids can be substituted or unsubstituted, straight chain, branched chain or cyclic (preferably straight chain), and saturated or unsaturated (mono- or poly-unsaturated) (preferably saturated). Non-limiting examples of suitable hydroxy acids include salicylic acid, glycolic acid, lactic acid, 5 octanoyl salicylic acid, hydroxyoctanoic acid, hydroxycaprylic acid, and lanolin fatty acids. Preferred concentrations of the organic hydroxy acid range from about 0.1% to about 10%, more preferably from about 0.2% to about 5%, also preferably from about 0.5% to about 2%. Salicylic acid is preferred. The organic hydroxy acids enhance the skin appearance benefits of the present invention. For example, the organic hydroxy acids tend to improve the texture of the skin.

A safe and effective amount of a desquamation agent can optionally be added to the cosmetic compositions of the subject invention. In some embodiments, desquamation agents/exfoliants can comprise from about 0.1% to about 10%, from about 0.2% to about 5%, or from about 0.5% to about 4% of the composition. Desquamation agents tend to improve the texture of the skin (e.g., smoothness). A variety of desquamation agents are known in the art and are suitable for use herein, including but not limited to the organic hydroxy agents described above.

The compositions of the present invention can also optionally include a safe and effective amount of a depilation agent. When used, the composition preferably contains from about 0.1% to about 10%, more preferably from about 0.2% to about 5%, also preferably from about 0.5% to about 2% of depilation agent. A depilation agent preferred for use herein comprises a sulfhydryl compound, e.g., N-acetyl-L-cysteine.

The compositions of the present invention can also optionally comprise a skin lightening agent. When used, the compositions preferably comprise from about 0.1% to about 10%, more preferably from about 0.2% to about 5%, also preferably from about 0.5% to about 2%, of a skin lightening agent. Suitable skin lightening agents include those known in the art, including kojic acid, arbutin, ascorbic acid and derivatives thereof, e.g., magnesium ascorbyl phosphate.

The cosmetic compositions of the present invention can also optionally comprise a zinc salt. Zinc salts are especially preferred where the composition contains a sulfhydryl compound, e.g., N-acetyl-L-cysteine. Without intending to be limited or bound by theory, it is believed that the zinc salt acts as a chelating agent capable of complexing with the sulfhydryl compound prior to topical application, stabilizes the sulfhydryl compound and/or controls odor associated with the sulfhydryl compound. Concentrations of the zinc salt can range from about 0.001% to about 10%, more preferably from about 0.01% to about 5%, most preferably from about 0.1% to about 0.5% by weight of the composition.

Preferred zinc salts include zinc acetate, zinc acetate hydrates such as zinc acetate-2-water, zinc aluminum oxide complexes such as gahnite, zinc diamine, zinc antimonide, zinc bromate hydrates such as zinc bromate-6water, zinc bromide, zinc carbonates such as zincspar and smithsonite, zinc chlorate hydrates such as zinc chlorate-4-water, zinc chloride, zinc diamine dichloride, zinc citrate, zinc chromate, zinc dichromate, zinc diphosphate, zinc hexacyanofluoride ferrate (II), zinc fluoride, zinc fluoride hydrates such as zinc fluoride-4-water, zinc formate, zinc formate hydrates such as zinc formate-2-water, zinc hydroxide, zinc iodate, zinc iodate hydrates such as zinc iodate-2-water, zinc iodide, zinc iron oxide complexes, zinc nitrate hydrates such as zinc nitrate-6-water, zinc nitride, zinc oxalate hydrates such as zinc oxalate-2-water, zinc oxides such as zincite, zinc perchlorate hydrates such as zinc perchlorate-6-water, zinc permanganate hydrates such as zinc permanganate-6-water, zinc peroxide, zinc p-phenolsulfonate hydrates such as zinc p-phenosulfonate-8-water, zinc phosphate, zinc phosphate hydrates such as zinc phosphate-4-water, zinc phosphide, zinc-propionate, zinc selenate hydrates such as zinc selenate-5-water, zinc selenide, zinc silicates such as zinc silicate (2) and zinc silicate (4), zinc silicon oxide water complexes such as hemimorphite, zinc hexafluorosilicate hydrates such as zinc hexafluorosilicate-6-water, zinc stearate, zinc sulfate, zinc sulfate hydrates such as zinc sulfate-7-water, zinc sulfide, zinc sulfite hydrates such as zinc sulfite-2-water, zinc telluride, zinc thiocyanate, zinc (II) salts of N-acetyl L-cysteine, and mixtures thereof.

The cosmetic compositions of the present invention can optionally further comprise a humectant, moisturizing agent or other skin conditioning agent. A variety of these materials can be employed and each can be present at a level of from or about 0.1% to or about 20%, more preferably from or about 1% to or about 10%, and most preferably from or about 2% to or about 5%. These materials include guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; polyethylene glycols; sugars and starches; sugar and starch derivatives (e.g., alkoxylated glucose); hyaluronic acid; lactamide monoethanolamine; acetamide monoethanolamine; and mixtures thereof. Also useful herein are the propoxylated glycerols described in U.S. Pat. No. 4,976,953, which is description is incorporated herein by reference.

Also optionally useful are various C₁-C₃₀ monoesters and polyesters of sugars and related materials. These esters are derived from a sugar or polyol moiety and one or more carboxylic acid moieties. Depending on the constituent acid and sugar, these esters can be in either liquid or solid form at room temperature. Examples of liquid esters include: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. A preferred solid material is sucrose polyester in which the degree of esterification is 7-8, and in which the fatty acid moieties are C:18 mono- and/or di-unsaturated and behenic, in a molar ratio of unsaturates:behenic of 1:7 to 3:5. A particularly preferred solid sugar polyester is the octaester of sucrose in which there are about 7 behenic fatty acid moieties and about 1 oleic acid moiety in the molecule. The ester materials are further described in, U.S. Pat. Nos. 2,831,854, 4,005,196, to Jandacek, issued Jan. 25, 1977; U.S. Pat. No. 4,005,195, to Jandacek, issued Jan. 25, 1977, U.S. Pat. No. 5,306,516, to Letton et al., issued Apr. 26, 1994; U.S. Pat. No. 5,306,515, to Letton et al., issued Apr. 26, 1994; U.S. Pat. No. 5,305,514, to Letton et al., issued Apr. 26, 1994; U.S. Pat. No. 4,797,300, to Jandacek et al., issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985; all of which are incorporated by reference herein in their entirety.

The cosmetic compositions of the present invention can also optionally include an extract obtained by suitable physical and/or chemical isolation from natural sources (e.g., plants), including those known in the topical personal care art. Preferred extracts are those which enhance the skin appearance benefits of the present invention, and which are preferably used in a safe and effective amount, more preferably an amount of from 0.1% to about 20%, even more preferably 0.5% to about 10%, also from 1% to about 5%. Such extracts include plant and fungal extracts such as extracts of yeast, rice bran, and of the plant Centella Asiatica. Natural extracts of Centella Asiatica are preferred and are commercially available from MMP, Inc. of Plainfield, N.J. under the trade name(s) Centella Asiatica E.P.C.A. (“Extract Purified of Centella asiatica”) and Genines amel. Genines amel is the purer form of the extract.

Compounds which are known to stimulate the production of collagen can also optionally be used in cosmetic composition of the present invention. Such compounds include Factor X (kinetin), Factor Z (zeatin), n-methyl taurine, dipalmitoyl hydroxyproline, palmitoyl hydroxy wheat protein, biopeptide CL (palmitoyl glycyl-histidyl-lysine), ASC III (Amplifier of Synthesis of Collagen III, E. Merck, Germany), and beta glucan.

The cosmetic compositions hereof can also optionally include natural ceramides or the like, for example, ceramide 1-6.

The cosmetic compositions can also optionally contain an oil absorbent such as are known in the art, e.g. clays (e.g. bentonite) and polymeric absorbents (e.g., Polymeric derivatised starches, (e.g., from National Starch), Derivatised globulin proteins, such as BioPol OE (Arch PC), MICROSPONGES 5647 and POLYTRAP, both commercially available from Advanced Polymer Systems, Inc. of Redwood City, Calif., USA., MICROSPONGES 5647 is a polymer mixture derived from styrene, methyl methacrylate, and hydrogel acrylate/methacrylate.

Other examples of additional components optionally useful herein include the following: water-soluble vitamins and derivatives thereof (e.g., vitamin C); polyethyleneglycols and polypropyleneglycols; polymers for aiding the film-forming properties and substantivity of the composition (such as a copolymer of eicosene and vinyl pyrrolidone, an example of which is available from GAF Chemical Corporation as Ganex.® V-220). Also useful are crosslinked and noncrosslinked nonionic and cationic polyacrylamides (e.g., Salcare SC92 which has the CTFA designation polyquaternium 32 (and) mineral oil, and Salcare SC 95 which has the CTFA designation polyquaternium 37 (and) mineral oil (and) PPG-1 trideceth-6, and the nonionic Seppi-Gel polyacrylamides available from Seppic Corp.). Also useful are crosslinked and uncrosslinked carboxylic acid polymers and copolymers such as those containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (examples useful herein include the carbomers, which are homopolymers of acrylic acid crosslinked with allyl ethers of sucrose or pentaerytritol and which are available as the Carbopol.® 900 series from B. F. Goodrich, and copolymers of C.sub.10-30 alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol, these copolymers being known as acrylates/C10-30 alkyl acrylate crosspolymers and are commercially available as Carbopol.® 1342, Pemulen TR-1, and Pemulen TR-2, from B. F. Goodrich). These carboxylic acid polymers and copolymers are more fully described in U.S. Pat. No. 5,087,445, to Haffey et al., issued Feb. 11, 1992; U.S. Pat. No. 4,509,949, to Huang et al., issued Apr. 5, 1985; U.S. Pat. No. 2,798,053, to Brown, issued Jul. 2, 1957; which are incorporated by reference herein. See also, CTFA International Cosmetic Ingredient Dictionary, fourth edition, 1991, pp. 12 and 80; which is also incorporated herein by reference.

### C. Saponification of Oil-Bearing Microbial Biomass and Extracted Oil

In some embodiments, microalgal biomass and/or microalgal oil can be combined with saponified oils derived from microalgae or other microorganisms. These saponified oils can optionally be used in place of soap components that may otherwise be combined with the microalgal biomass or microalgal oil to form cosmetic compositions in accordance with the present invention. In some cases, a portion of a the microalgal oil (triacylglycerides) is saponified, and the partially saponified oil is combined with one or more other cosmetic ingredients to form a cosmetic compositions including both saponified microalgal oil and non-saponified microalgal oil. As described below, the ratio of saponified oil to non-saponified oil can be modified by controlling the quantity of base used in the reaction.

Animal and plant oils are typically made of triacylglycerols (TAGs), which are esters of fatty acids with the trihydric alcohol, glycerol. In an alkaline hydrolysis reaction, the glycerol in a TAG is removed, leaving three carboxylic acid anions that can associate with alkali metal cations such as sodium or potassium to produce fatty acid salts. A typical reaction scheme is as follows:

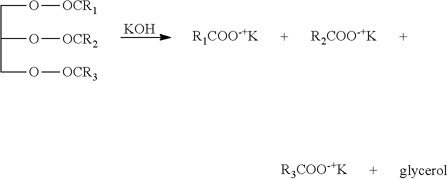

In this scheme, the carboxylic acid constituents are cleaved from the glycerol moiety and replaced with hydroxyl groups. The quantity of base (e.g., KOH) that is used in the reaction is determined by the desired degree of saponification. If the objective is, for example, to produce a soap product that comprises some of the oils originally present in the TAG composition, an amount of base insufficient to convert all of the TAGs to fatty acid salts is introduced into the reaction mixture. Normally, this reaction is performed in an aqueous solution and proceeds slowly, but may be expedited by the addition of heat. Precipitation of the fatty acid salts can be facilitated by addition of salts, such as water-soluble alkali metal halides (e.g., NaCl or KCl), to the reaction mixture. Preferably, the base is an alkali metal hydroxide, such as NaOH or KOH. Alternatively, other bases, such as alkanolamines, including for example triethanolamine and aminomethylpropanol, can be used in the reaction scheme. In some cases, these alternatives may be preferred to produce a clear soap product.

Saponification of oil bearing microbial biomass can be performed on intact biomass or biomass that has been disrupted prior to being subjected to the alkaline hydrolysis reaction. In the former case, intact microbial biomass generated via the culturing of microorganisms as described herein can be directly contacted with a base to convert ester-containing lipid components of the biomass to fatty acid salts. In some cases, all or a portion of the water in which the microbes have been cultured is removed and the biomass is resuspended in an aqueous solution containing an amount of base sufficient to saponify the desired portion of the glycerolipid and fatty acid ester components of the biomass. In some cases, less than 100% of the glycerolipids and fatty acid esters in the biomass are converted to fatty acid salts.

In some methods of the invention, the biomass is disrupted prior to being subjected to the alkaline hydrolysis reaction. Disruption of the biomass can be accomplished via any one or more of the methods described above for lysing cells, including heat-induced lysis, mechanical lysis, or the like, in order to make the intracellular contents of the microorganisms more readily accessible to the base. This can help to facilitate the conversion of TAGs or fatty acid esters to fatty acid salts. Although acid-induced lysis can be used to disrupt the biomass prior to saponification, other methods may be more desirable to reduce the possibility that additional base will be consumed to neutralize any remaining acid during the alkaline hydrolysis reaction, which may impact the conversion efficiency to fatty acid salts. Because the application of heat can expedite the alkaline hydrolysis reaction, heat-induced lysis can be used prior to or during the saponification reaction to produce the fatty acid salts.

In some embodiments, the biomass is not subjected to any treatment, or any treatment other than disruption, prior to being subjected to the alkaline hydrolysis reaction. In some embodiments, prior enrichment of the biomass to increase the ratio of lipid to non-lipid material in the biomass to more than 50% (or by more than 50%) by weight, is performed. In other embodiments, the biomass is subjected to the alkaline hydrolysis reaction without a step of prior enrichment. In some cases, the biomass subjected to the alkaline hydrolysis reaction contains components other than water in the same relative proportions as the biomass at the point of harvesting. In those cases in which substantially all of the water has been removed, the biomass comprises a cellular emulsion or substantially-dried emulsion concentrate.

Any of the microorganisms described herein can be used to produce lipid-containing biomass for the production of saponified oils. In some cases, the microorganisms can also impart other characteristics to the saponified-oil compositions produced from the methods described herein. For example, microalgae of different species, as well as microalgae grown under different conditions, vary in color, including green, yellow, orange, red, and the like. Small quantities of the compounds that impart these colors to the microalgae can contaminate (e.g., by purposefully retaining some of these materials) the resulting saponified-oil compositions and thereby provide natural colorants. In some cases, other constituents of the biomass, including carotenoids and xanthophylls, can also be retained in small quantities in the saponified-oil compositions.

The extent of saponification of the biomass can vary in the methods of the invention. In some cases it is desirable to produce a saponified-oil composition that also includes glycerolipid constituents of the biomass. The appropriate quantity of base (e.g., NaOH) for use in the alkaline hydrolysis reaction can be determined based on an analysis of the glycerolipid and fatty acid ester content of the biomass. In some cases, it is preferable to use an excess of base to directly saponify lipid-containing biomass because some of the base may be consumed by reaction with other constituents of the biomass. In some cases, the use of excess quantities of base to saponify the ester-containing lipid constituents of the biomass results in a saponified oil composition that is undesirably alkaline. In these instances, the composition can be purified to reduce the alkalinity of the composition by boiling the saponified oil composition in water and re-precipitating the fatty acid salts via addition of salts such as NaCl, KCl, or the like. The purified soap composition can then be subjected to further processing, such as removing excess water, introducing various additives into the soap composition, moulding the soap in bars or other shapes, or the like.

In some cases, the fatty acid salts (also referred to as saponified oils) generated from the methods described herein can be combined with microalgal biomass, microalgal oil, and/or other cosmetic ingredients as described herein.

The degree of saponification of extracted lipid constituents of the biomass is more readily controlled because of a reduced probability that the base will be consumed through interaction with components other than glycerolipids or fatty acid esters present in the extracted oil. Extraction of the lipid constituents can be performed via conventional hexane-extraction procedures, or via an oil-extraction or solventless-extraction procedure.

Conventional hexane-extraction (other suitable organic solvents can also be used) generally comprises contacting the biomass or lysate with hexane in an amount and for a period of time sufficient to allow the lipid to form a solution with the hexane. The mixture can then be filtered and the hexane removed by, for example, rotoevaporation. Hexane extraction methods are well known in the art.

Oil extraction includes the addition of an oil directly to a lysate without prior separation of the lysate components. After addition of the oil, the lysate separates either of its own accord or as a result of centrifugation or the like into different layers. The layers can include in order of decreasing density: a pellet of heavy solids, an aqueous phase, an emulsion phase, and an oil phase. The emulsion phase is an emulsion of lipids and aqueous phase. Depending on the percentage of oil added with respect to the lysate (w/w or v/v), the force of centrifugation if any, volume of aqueous media and other factors, either or both of the emulsion and oil phases can be present. Incubation or treatment of the cell lysate or the emulsion phase with the oil is performed for a time sufficient to allow the lipid produced by the microorganism to become solubilized in the oil to form a heterogeneous mixture.

In various embodiments, the oil used in the extraction process is selected from the group consisting of oil from soy, rapeseed, canola, palm, palm kernel, coconut, corn, waste vegetable oil, Chinese tallow, olive, sunflower, cotton seed, chicken fat, beef tallow, porcine tallow, microalgae, macroalgae, Cuphea, flax, peanut, choice white grease (lard), Camelina sativa mustard seedcashew nut, oats, lupine, kenaf, calendula, hemp, coffee, linseed, hazelnut, euphorbia, pumpkin seed, coriander, camellia, sesame, safflower, rice, tung oil tree, cocoa, copra, pium poppy, castor beans, pecan, jojoba, jatropha, macadamia, Brazil nuts, and avocado. The amount of oil added to the lysate is typically greater than 5% (measured by v/v and/or w/w) of the lysate with which the oil is being combined. Thus, a preferred v/v or w/w of the oil is greater than 5%, or at least 6%, at least 7%, at least 10%, at least 20%, at least 25%, at least 30%, at least 40%, at least 50%, at least 60%, at least 70%, at least 80%, at least 90%, and at least 95% of the cell lysate.

Lipids can also be extracted from a lysate via a solventless extraction procedure without substantial or any use of organic solvents or oils by cooling the lysate. In such methods, the lysate is preferably produced by acid treatment in combination with above room temperature. Sonication can also be used, particularly if the temperature is between room temperature and 65° C. Such a lysate on centrifugation or settling can be separated into layers, one of which is an aqueous:lipid layer. Other layers can include a solid pellet, an aqueous layer, and a lipid layer. Lipid can be extracted from the emulsion layer by freeze thawing or otherwise cooling the emulsion. In such methods, it is not necessary to add any organic solvent or oil. If any solvent or oil is added, it can be below 5% v/v or w/w of the lysate.

The extracted lipids are then subjected to an alkaline hydrolysis reaction as described above, in which the amount of base added to the reaction mixture can be tailored to saponify a desired amount of the glycerolipid and fatty acid ester constituents of the lipid composition. A close approximation or quantification of the amount of esterified lipid in the composition can be used to tailor the amount of base needed to saponify a specified portion of the oil, thereby providing an opportunity to modulate the amount of unsaponified oil remaining in the resulting composition. In some cases, at least 1%, at least 2%, at least 3%, at least 4%, at least 5%, at least 6%, at least 7%, at least 8%, at least 9%, or at least 10% of the oil, by weight, remains unsaponified in the resulting composition. In other cases, it may be desirable to saponify all or substantially all of the oil, such that the resulting composition contains no more than 10%, no more than 9%, no more than 8%, no more than 7%, no more than 6%, no more than 5%, no more than 4%, no more than 3%, no more than 2%, no more than 1%, or no more than 0.5% unsaponified oil by weight.

In various embodiments of the invention, the microbial biomass can contain lipids with varying carbon chain lengths, and with varying levels of saturation. The characteristics of the lipids can result from the natural glycerolipid profiles of the one or more microorganism populations used to generate the biomass subjected to the saponification reaction, or can be the result of lipid pathway engineering, as described herein, in which transgenic strains of microorganisms are designed to produce particular lipids in greater proportions.

### D. Cosmetic Compositions of Microalgal Biomass and Algal Oil

In one aspect, the present invention is directed to cosmetic compositions comprising at least 1% w/w microalgal biomass and/or microalgal oil. In some embodiments, the cosmetic compositions comprise at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% microalgal biomass and/or microalgal oil. The remainder of a cosmetic composition in accordance with the present invention comprises water or other conventional cosmetic ingredients, including those identified herein.

Cosmetic compositions of the present invention can be in the form of finished cosmetic products for use in skin care, bathing, and/or other applications pertaining to the maintenance or improvement of an individual's appearance or health. In other cases, the cosmetic compositions of the invention are in the form of cosmetic ingredients themselves, for use in combination with other cosmetic ingredients in the production of finished cosmetic products.

In some embodiments, cosmetic compositions of the present invention comprise at least 1% w/w microalgal biomass, or a greater percentage as described above. The microalgal biomass comprises at least 10% microalgal oil by dry weight, and can include greater amounts of microalgal oil as well as other constituents as described herein.

The microalgal biomass useful in the cosmetic compositions of the invention can be derived from one or more species of microalgae cultured and/or genetically engineered as described herein.

In various embodiments, cosmetic compositions comprising microalgal biomass can be formulated as decorative or care cosmetics with one or more other cosmetic ingredients. Exemplary cosmetic compositions include, without limitation, skin-care creams, lotions, powders, perfumes and deodorants, lipsticks, bath oils, bath scrubs and cleansing products, masks, and the like.

In some embodiments, cosmetic compositions of the present invention comprise at least 1% w/w microalgal oil, or a greater percentage as described above. The microalgal oil is derived from cultures of microalgae grown under heterotrophic conditions or those comprising at least 10% oil by dry cell weight, as described herein. In some cases, the microalgae can be genetically engineered.

In various embodiments, cosmetic compositions comprising microalgal oil can be formulated as decorative or care cosmetics with one or more other cosmetic ingredients. Exemplary cosmetic compositions include, without limitation, skin-care creams, lotions, beauty oils, perfumes and deodorants, lipsticks, bath oils, bath scrubs and cleansing products, masks, and the like.

### E. Use in Conventional Finished Cosmetic Products

In some cases, microalgal cosmetic compositions in accordance with the present invention can be used in otherwise conventional finished cosmetic products. In these instances, the cosmetic composition comprising microalgal biomass and/or microalgal oil is combined with one or more other cosmetic ingredients, as described herein, to form a cosmetic composition that may be packaged as a finished cosmetic product. In some cases, microalgal cosmetic compositions of the present invention can be packaged as a cosmetic ingredient with optional instructions for combining the microalgal composition with conventional cosmetic ingredients to create finished cosmetic products.

In at least one embodiment, the present invention is directed to a method of preparing a finished cosmetic composition, e.g., a skin-care product, comprising (i) culturing a population of microalgae under conditions to generate microalgal biomass comprising at least 10% microalgal oil by dry weight, (ii) harvesting the biomass from the microalgal culture, (iii) performing one or more optional processing steps, e.g., drying the biomass or extracting oil from the biomass, (iv) combining the biomass or the extracted oil with at least one other cosmetic ingredient to form a cosmetic composition, and (v) packaging the cosmetic composition with optional instructions for its use as a finished cosmetic product.

In one aspect, the present invention is directed to a method of using a microalgal biomass composition to soften and impart pliability to skin. In one embodiment, the microalgal biomass composition comprises predominantly intact microalgal cells containing at least 10% microalgal oil by dry weight. Preferably, the algal oil present in the composition is predominantly encapsulated in cells of the biomass. The microalgal biomass composition is applied to human skin and retained in contact with the skin for a period of time sufficient to permit release of a specified percentage of the oil from the intact microalgal cells by enzymatic degradation of the cells. For example, the composition can be retained in contact with the skin for a period of time sufficient to release at least 50% of the microalgal oil from the predominantly intact cells. In some cases, this period may be from 3-4 hours.

Without intending to be bound by any particular theory, it is believed that enzymes present on human skin will slowly degrade the intact microalgal cells, thereby releasing the intracellular contents, including microalgal oil, over a period of time. In some embodiments, the microalgal biomass composition is retained in contact with the skin for at least 15 minutes, for at least 30 minutes, for at least 45 minutes, for at least 1 hour, for at least 2 hours, for at least 3 hours, or for at least 4 hours or more.

Microalgal biomass compositions useful in the above method can also comprise cells containing at least 25%, at least 35%, or at least 45% oil by dry weight. In other cases, the cells may contain other percentages of oil as described herein. In some cases, mixtures of microalgal cells having different glycerolipid profiles can be combined together to form the microalgal biomass composition.

All references cited herein, including patents, patent applications, and publications, are hereby incorporated by reference in their entireties, whether previously specifically incorporated or not. The publications mentioned herein are cited for the purpose of describing and disclosing reagents, methodologies and concepts that may be used in connection with the present invention. Nothing herein is to be construed as an admission that these references are prior art in relation to the inventions described herein. In particular, the following patent applications are hereby incorporated by reference in their entireties for all purposes: U.S. Provisional Application No. 61/074,610, filed Jun. 20, 2008, entitled “Soaps and Cosmetics Products Produced from Oil-Bearing Microbial Biomass and Oils”; U.S. Provisional Application No. 61/105,121, filed Oct. 14, 2008, entitled “Food Compositions of Microalgal Biomass”; PCT Patent Application No. PCT/US2008/065563, filed Jun. 2, 2008, entitled “Production of Oil in Microorganisms”; PCT Patent Application No. PCT/US2007/001653, filed Jan. 19, 2007, entitled “Microalgae-Derived Composition for Improving Health and Appearance of Skin”; and U.S. patent application Ser. No. 12/176,320, filed Jul. 18, 2008, entitled “Compositions for Improving the Health and Appearance of Skin”.

### F. Anti-Aging Repairing Formula

In an embodiment of the present invention, an anti-aging repairing formula for topical application to the skin, and especially to the face, is formulated with a microalgal oil. In a specific embodiment, the oil is produced by heterotrophic cultivation of Chlorella or Chlorella protothecoides. The oil can be combined with one or more of a lubricant, a binder, a thinner, a moisturizer, a dermal cell-signaling molecule, an elastin inhibitor, an antioxidant, a retinoid, and a fragrance. In a specific embodiment, Chlorella oil is combined with a retinoid and one or more of a ceramide, alaria esculenta extract, rosemary extract, tocopherol, and cympogon martini oil.

In a specific embodiment, the formula comprises oil extracted from Chlorella protothecoides (predominantly triglyceride and sterols), cetearyl ethylhexanoate, isopropyl isostearate, caprylic/capric triglyceride, ceramide (e.g., ceramide 3), Alaria Esculent Extract, Rosemary extract, tocopherol(s), retinyl palmitate, and Cymphogon martini oil. Optionally, these are combined in the following proportions:

| Ingredient | Amount (% wt/wt) |
|---|---|
| Oil extracted from | 10-50% |
| Chlorella protothecoides | |
| Cetearyl ethylhexanoate | 20-40% |
| Isopropyl isostearate | 10-40% |
| Caprylic/Capric Triglyceride | 5-20% |
| Ceramide 3 | 0.001-0.02 |
| Alaria Esculent Extract (with | 0.1-2.0% |
| Caprylic/Capric Triglyceride) | |
| Rosemary extract (in vegetable | 0.01-0.2% |
| oil) | |
| DL-alpha tocopherol | 0.01-0.2% |
| Retinyl palmitate | 0.01-0.2% |
| Cymphogon martini oil | 0.01-0.2% |

### EXAMPLES

The following examples are offered to illustrate, but not to limit, the claimed invention.

### Example 1

In the following examples and tables, strains were prepared and grown heterotrophically as described above and in WO2008/151149, WO2010/063031, WO2010/045368, WO2010/063032, WO2011/150411, WO2013/158938, 61/923,327 filed Jan. 3, 2014, PCT/US2014/037898 filed May 13, 2014, and in U.S. Pat. No. 8,557,249. Sample A refers to oil from Chlorella (Auxeochlorella) protothecoides cells (UTEX 250). WAF refers to whole algal flour and WAP refers to whole algal protein and are Chlorella (Auxeochlorella) protothecoides cells (UTEX 250) cultivated for lipid and high protein content. CF refers to Chlorella (Auxeochlorella) protothecoides cells (UTEX 250) classically mutagenized and selected for color loss (S1330). Samples B-F are oil isolated from various strains originating from Prototheca moriformis (UTEX 1435) that were prepared and cultured to achieve the indicated fatty acid profile. Sample H oil used in Examples 8 and 9 has a fatty acid profile of approximately 26% C16:0, 5% C18:0, 59% C18:1, 5% C18:2, and less than 0.5% C18:3 and is extracted from a classically mutagenized strain of Prototheca moriformis (UTEX 1435). UTEX 250 and 1435 are available from the University of Texas at Austin Culture Collection of Algae.

TABLE I
Oil properties

| | | Sample | | | | | | |
|---|---|---|---|---|---|---|---|---|
| Assay | | A (UTEX | B (high | C | D (high | E | F (low poly- | G |
| Fatty Acid Profile | Units | 250) | C10-C12) | (laurate) | myristic) | (SOS) | unsaturates) | (Oleic) |
| C8:0 | % | 0.00 | 1.02 | 0.35 | 0.00 | 0.00 | 0.00 | 0.00 |
| C10:0 | % | 0.08 | 40.45 | 18.18 | 0.04 | 0.03 | 0.03 | 0.01 |
| C12:0 | % | 0.22 | 45.00 | 45.92 | 0.89 | 0.19 | 0.06 | 0.03 |
| C14:0 | % | 1.29 | 4.00 | 12.92 | 56.94 | 0.47 | 0.35 | 0.41 |
| C16:0 | % | 17.44 | 2.33 | 6.34 | 14.98 | 3.03 | 3.29 | 3.31 |
| C18:0 | % | 1.66 | 0.27 | 0.51 | 0.68 | 56.75 | 2.87 | 2.22 |
| C18:1 | % | 59.12 | 4.24 | 10.12 | 20.51 | 33.90 | 89.94 | 86.17 |
| C18:2 | % | 15.17 | 1.62 | 3.32 | 4.26 | 1.94 | 1.03 | 5.50 |
| C18:3 ALPHA | % | 2.01 | 0.27 | 0.38 | 0.23 | 0.16 | 0.15 | 0.24 |
| C20:0 | % | 0.25 | 0.02 | 0.06 | 0.06 | 1.65 | 0.25 | 0.26 |
| DROPPING MELTING | ° C. | 10.5 | 22.2 | 27.2 | | | 2 | 0.3 |
| POINT (METTLER) | | | | | | | | |
| AOCS Cc 18-80 | | | | | | | | |
| CLOUD POINT D97 | ° C. | | 12 | 17 | 29 | | −18 | −19 |
| POUR POINT D97 | ° C. | | 10 | 15 | 27 | | −20 | −21 |
| IODINE VALUE | unit | 85.6 | 8.8 | 18.7 | 27.7 | | 81.6 | 85.6 |
| OSI RANCIMAT | hours | | 68.72 | 46.8 | 37.56 | | 57.6 | 19.35 |
| (110° C.) AOCS Cd | | | | | | | | |
| 12b-92 | | | | | | | | |
| SMOKE POINT AOCS | ° C. | | | | 150 | | 248 | 248 |
| Cc 9a-48 | | | | | | | | |
| SAPONIFICATION | mg | | | 239.2 | | | | |
| VALUE AOCS Cd 3-25 | KOH/g | | | | | | | |
| ALPHA | mg/100 g | 12.7 | — | | 0.22 | | — | — |
| TOCOPHEROL | | | | | | | | |
| B-SITOSTEROL | mg/100 g | 56.3 | — | | 6.51 | | 26.4 | 3.81 |
| BETA TOCOPHEROL | mg/100 g | — | — | | — | | — | — |
| BRASSICASTEROL | mg/100 g | 131 | — | | — | | — | — |
| CAMPESTEROL | mg/100 g | 16.8 | 11.9 | | 6.29 | 3.72 | 8.03 | 8.08 |
| CHOLESTEROL | mg/100 g | — | — | | — | | — | — |
| DELTA TOCOPHEROL | mg/100 g | 5.47 | 0.76 | | 0.28 | 1.48 | — | 0.81 |
| ERGOSTEROL | mg/100 g | 130 | 59.2 | | 174 | 54.8 | 174 | 92 |
| GAMMA | mg/100 g | 2.25 | — | | 0.28 | 0.83 | 0.57 | 0.12 |
| TOCOPHEROL | | | | | | | | |
| STIGMASTEROL | mg/100 g | 18.7 | 6.19 | | 16.3 | 13.3 | 15.7 | 11.6 |
| OTHER STEROLS | mg/100 g | 279 | 111 | | 151 | 139 | 98.3 | 130 |
| ALPHA | mg/g | 0.11 | | | 0.18 | 0.17 | | |
| TOCOTRIENOL | | | | | | | | |
| BETA TOCOTRIENOL | mg/g | 0.02 | | | 0.04 | <0.01 | | |
| DELTA | mg/g | 0.06 | | | <0.01 | <0.01 | | |
| TOCOTRIENOL | | | | | | | | |
| GAMMA | mg/g | 0.02 | | | 0.03 | 0.07 | | |
| TOCOTRIENOL | | | | | | | | |
| TOTAL | mg/g | 0.21 | | | 0.25 | 0.24 | | |
| TOCOTRIENOLS | | | | | | | | |

### Example 2

A fibroblast cell culture model was used to assess the effect of whole algal flour (WAF), whole algal protein (WAP), whole algal protein extract, high C10-12 oil (Sample B), and high-stability oil (Sample F) on procollagen, elastin, and hyaluronic acid synthesis. The type I C-peptide fragment was measured because procollagen, a precursor to mature collagen, is cleaved in collagen and type I C-peptide fragments in a 1:1 ratio. By measuring the amount of type I C-peptide fragments in the tissue medium, collagen production was measured. Elastin was measured directly from the cell culture medium. Hyaluronic acid was measured in the tissue culture media. All measurements were performed using an ELISA based method.

Further, changes in cell number were assessed via an MTT assay. The MTT analysis involved a colorimetric analysis of tissue metabolic activity, the increased presence of color indicating the presence of metabolically-active living cells. Reduction of MTT by mitochondria in viable cells results in purple formazin crystals; the intensity of the purple color is directly proportional to metabolically active cells in the tissue.

Powder materials were prepared by combining 1 gram of the powder material with either 10 ml ultrapure water or 10 ml of DMSO. The mixtures were incubated for one hour at room temperature on a rocking platform to saturate the extraction medium with the test material. The mixture was then centrifuged to separate the saturated extract solution from the undissolved solid materials. The oil based materials were prepared as a stock solution in DMSO and then diluted into the culture media such that the final concentration of DMSO was 1%. A 1% DMSO control was included to account for any effect the solvent might have when evaluating the effect of the oils or DMSO prepared powders.

The fibroblast cell culture was prepared by seeding fibroblasts into the individuals wells of a 24-well plate in 0.5 ml of Fibroblast Growth Media (FGM) and incubated overnight at 37±2° C. and at 5±1% CO₂. The media was removed via aspiration to eliminate non-adherent cells and replaced with 0.5 ml of fresh FGM. The medium was changed every 48 to 72 hours until the cells growth reached confluency. Once confluent, the cells were treated for 24 hours with DMEM supplemented with 1.5% FBS to wash out any growth factors present in the normal culture media.

The prepared fibroblast cell cultures were then treated with the prepared test materials at the aforementioned concentrations dissolved in FGM with 1.5% FBS. TGF-B at a concentration of 50 ng/ml was used as a positive control for collagen and elastin, and dibutyrl cAMP at a concentration of 0.1 mM was used as a positive control for hyaluronic acid. Untreated cells received DMEM with 1.5% FBS. The cells were incubated for 48 hours and then either frozen or assayed immediately. Materials were tested in triplicate.

After the 2-day incubation an MTT assay was performed. The cell culture medium was removed and the fibroblasts were washed twice with PBS to remove any remaining test material. After the final wash, 500 μl of DMEM supplemented with 0.5 mg/ml MTT was added to each well and the cells were incubated for 1 hour at 37±2° C. and 5±1% CO₂. After the incubation, the DMEM/MTT solution was removed and the cells were washed again once with PBS and then 0.5 ml of isopropyl alcohol was added to the well to extract the purple formazin crystals. Two hundred microliters of the isopropyl extracts was transferred to a 96-well plate and the plate was read at 540 nm using isopropyl alcohol as a blank.

TABLE II
MTT Assay

| Treatment | Percent Viability |
|---|---|
| Untreated | 100 ± 2.2 |
| 50 ng/ml TGF-B | 111 ± 1.1 |
| 0.1 mM dBcAMP | 128 ± 0.0 |
| 1% DMSO | 121 ± 2.7 |
| 20% WAF | 117 ± 4.1 |
| 10% WAF | 117 ± 4.4 |
| 1% WAF | 110 ± 3.5 |
| 1% WAF DMSO | 99 ± 4.8 |
| 10% WAP | 150 ± 0.5 |
| 1% WAP | 109 ± 0.5 |
| 1% WAP DMSO | 103 ± 1.0 |
| 10% CF | 135 ± 0.5 |
| 1% CF | 113 ± 2.8 |
| 1% CF DMSO | 117 ± 3.5 |
| 0.05% Sample F | 105 ± 4.0 |
| 0.05% Sample B | 118 ± 9.3 |

A series of type I C-peptide standards were prepared ranging from 0 ng/ml to 640 ng/ml for the procollagen assay. Next, an ELISA microplate was prepared by removing any unneeded strips from the plate frame followed by the addition of 100 μl of peroxidase-labeled anti-procollagen type I-C peptide antibody to each well used in the assay. 20 μl of either sample (collected tissue culture media) or standard was then added to appropriate wells and the microplate was covered and allowed to incubate for 3±0.25 hours at 37° C. After incubation, the wells were aspirated and washed three times with 400 μl of wash buffer. After the last wash was removed 100 μl of peroxidase substrate solution (hydrogen peroxide+tetramethylbenzidine as a chromagen) was added to each well and the plate was incubated for 10-20 minutes at room temperature. After the incubation 100 μl of stop solution (1 N sulfuric acid) was added to each well and the plate was read using a microplate reader at 450 nm.

TABLE III
Type I Collagen Assay

| Treatment | Type I C-Peptide (ng/ml) |
|---|---|
| Untreated | 3212 ± 294 |
| 50 ng/ml TGF-B | 5282 ± 178 |
| 0.1 mM dBcAMP | 3438 ± 277 |
| 1% DMSO | 2882 ± 366 |
| 20% WAF | 2295 ± 307 |
| 10% WAF | 1945 ± 354* |
| 1% WAF | 2044 ± 293* |
| 1% WAF | 2760 ± 294 |
| 10% WAP | 3383 ± 199 |
| 1% WAP | 2701 ± 733 |
| 1% WAP DMSO | 4116 ± 365*, ** |
| 10% CF | 2382 ± 238 |
| 1% CF | 2555 ± 521 |
| 1% CF DMSO | 2658 ± 315 |
| 0.05% Sample F | 4066 ± 527*, ** |
| 0.05% Sample B | 3131 ± 226 |

*Denotes values that are significantly different from the Untreated group (p < 0.05).
**Denotes values that are significantly different from 1% DMSO (p < 0.05).

In preparing the elastin ELISA plate, soluble alpha-elastin was dissolved in 0.1 M sodium carbonate (pH 9.0) at a concentration of 1.25 μg/ml. 150 μl of this solution was then applied to the wells of a 96-well maxisorp Nunc plate and the plate was incubated overnight at 4° C. On the following day, the wells were saturated with PBS containing 0.25% BSA and 0.05% Tween 20. The plate was then incubated with this blocking solution for 1 hour at 37° C. and then washed two times with PBS containing 0.05% Tween 20.

A set of alpha-elastin standards was generated ranging from 0 to 100 ng/ml. 180 μl of either standard or sample was then transferred to a 650 μl microcentrifuge tube. An anti-elastin antibody solution was prepared (the antibody was diluted 1:100 in PBS containing 0.25% BSA and 0.05% Tween 20) and 20 μl of the solution was added to the tube. The tubes were then incubated overnight at 4±2° C. On the following day, 150 μl was transferred from each tube to the 96-well elastin ELISA plate, and the plate was incubated for 1 hour at room temperature. The plate was then washed 3 times with PBS containing 0.05% Tween 20. After washing, 200 μl of a solution containing a peroxidase linked secondary antibody diluted in PBS containing 0.25% BSA and 0.05% Tween 20 was added, and the plate was incubated for 1 hour at room temperature. After washing the plate three times as described above, 200 μl of a substrate solution was added and the plate was incubated for 20±10 minutes in the dark at room temperature. After the final incubation, the plate was read at 460 nm using a plate reader.

TABLE IV
Elastin Assay

| Treatment | Elastin (ng/ml) |
|---|---|
| Untreated | 17 ± 2 |
| 50 ng/ml TGF-B | 178 ± 6* |
| 0.1 mM dBcAMP | 43 ± 16 |
| 1% DMSO | 28 ± 13 |
| 20% WAF | 18 ± 8 |
| 10% WAF | 13 ± 4 |
| 1% WAF | 35 ± 12 |
| 1% WAF DMSO | 46 ± 6* |
| 10% WAP | 16 ± 2 |
| 1% WAP | 32 ± 6 |
| 1% WAP DMSO | 33 ± 2 |
| 10% CF | 30 ± 6 |
| 1% CF | 39 ± 11* |
| 1% CF DMSO | 33 ± 6 |
| 0.05% Sample F | 51 ± 5*, ** |
| 0.05% Sample B | 38 ± 4 |

*Denotes values that are significantly different from the Untreated group (p < 0.05).
**Denotes values that are significantly different from 1% DMSO (p < 0.05).

A series of hyaluronic acid standards was prepared ranging from 50 ng/ml to 3,200 ng/ml. Next, 100 μl of each standard (in duplicate) and sample was transferred to a well in an incubation plate. After adding 50 μl of detection solution to each well (except the reagent blank wells) the plate was incubated for 1±0.25 hour at 37±2° C. After the incubation, 100 μl of each sample/standard from the incubation plate was transferred to a corresponding well in the ELISA plate. The ELISA plate was covered and incubated for 30±5 minutes at 4° C. and then washed three times with 300 μl of wash buffer. After the final wash 100 μl of enzyme solution was added to each well and the plate was incubated at 37±2° C. for 30±5 minutes. After this incubation the wells were washed again as described above and then 100 μl of enzyme substrate solution was added to each well and the plate was incubated for 30-45 minutes at room temperature. After this final incubation 50 μl of stop solution was added to each well and the absorbance of the plate was measured at 405 nm using a plate reader.

TABLE Va
Hyaluronic Acid Assay

| Treatment | Hyaluronic Acid (ng/ml) |
|---|---|
| Untreated | 72 ± 22 |
| 50 ng/ml TGF-B | 260 ± 34 |
| 0.1 mM dBcAMP | 494 ± 51 |
| 1% DMSO | 23 ± 1 |
| 20% WAF extract | 154 ± 7 |
| 10% WAF extract | 193 ± 31 |
| 1% WAF extract | 85 ± 4 |
| 1% WAF DMSO extract | 22 ± 1 |
| 10% WAP extract | 3313 ± 313 |
| 1% WAP extract | 203 ± 9 |
| 1% WAP DMSO extract | 24 ± 8 |
| 10% CF extract | 177 ± 61 |
| 1% CF extract | 157 ± 23 |
| 1% CF DMSO extract | 38 ± 3 |
| 0.05% Sample F oil | 34 ± 7 |
| 0.05% Sample B oil | 32 ± 2 |

TABLE Vb
Hyaluronic Acid Assay

| Treatment | Hyaluronic Acid (ng/ml) |
|---|---|
| Untreated | 187 ± 24 |
| 50 ng/ml TGF-B | 528 ± 96 |
| 0.1 mM dBcAMP | 547 ± 31 |
| 1% WAP | 1398 ± 8 |
| 0.5% WAP | 1245 ± 73 |
| 0.1% WAP | 843 ± 79 |

A fibroblast cell culture model was used to assess the ability of the test materials to exert an effect on procollagen, elastin, and hyaluronic acid synthesis. This study also assessed the viability of the cells after exposure to the test materials. With respect to collagen synthesis, two of the test materials were observed to significantly increase this endpoint. These two materials were 1% WAP DMSO extraction and the 0.05% Sample F oil. For elastin, three materials were observed to significantly increase this endpoint. These materials were 1% WAF DMSO extract, 1% CF and the 0.05% Sample F oil again. Finally, for hyaluronic acid synthesis, one of the materials produced a very dramatic increase in this endpoint. This material was the 10% WAP extract material. Additionally, whole cells were tested in the hyaluronic acid assay (Table Va) and were also found to have a statistically significant (p<0.05) increase in hyaluronic acid production.

There was a question regarding the dramatic increase in hyaluronic acid synthesis with the WAP extract material since this material contains materials similar enough to hyaluronic acid to cross react with the ELISA kit. However, when the test material was prepared in culture media and assayed (without being applied to cells), the hyaluronic acid in detected in these samples was close to background levels found in the media (approximately 31-36 ng/ml) and well below the levels produced when the fibroblasts were treated with the test material. Thus the strong production of hyaluronic acid observed in this study was due to an effect of the material on the cultured fibroblasts and not due to any cross reaction of the test material with the assay.

### Example 3

A UVB protection assay was prepared to analyze the efficacy of whole algal flour (WAF), Sample B, high-stability oil (Sample F), and cosmetic algal oil (Sample A) in reducing tissue viability after exposure to UVB radiation. MatTek's EpiDerm tissue, a skin model that consists of normal human-derived epidermal keratinocytes cultured to form a multilayered, highly differentiated model of the human epidermis, was used to measure changes in tissue viability. The tissues were exposed to 300 mJ/cm² of UVB radiation and subsequently topically treated with the aforementioned test materials over a period of 24 hours. Materials were tested for their abilities to either maintain tissue viability after UVB exposure or aid in the recovery of tissue damaged by UVB radiation.

To measure changes in tissue viability, a MTT (3-(4,5-Dimethylthiazol-2-yl)-2,5-diphenyltetrazolium bromide, a tetrazole) assay was performed. The MTT analysis involved a colorimetric analysis of tissue metabolic activity, the increased presence of color indicating the presence of metabolically-active living cells. Reduction of MTT by mitochondria in viable cells results in purple formazin crystals; the intensity of the purple color is directly proportional to metabolically active cells in the tissue.

The tissues were stored at 4° C. until used. Before use, the tissues were placed in 0.9 ml of assay medium and allowed to incubate for at least 1 hour at 37±2° C. and at 5±1% CO₂. One gram of WAF was mixed with 10 ml of water for one hour at room temperature. The WAF-water mixture was then centrifuged for 5 minutes to pellet any undissolved material and the supernatant was used for topical application. The Sample B, Sample F, and Sample A were applied topically without any further processing. Mineral oil served as a control for the oil based materials to account for any non-specific effects of oils on UVB protection. After the UVB exposure 100 μl of test material, mineral oil alone (oil control), 1 mM trolox (positive control), or PBS alone (negative control) was applied to the tissues and the tissues were incubated for 24 hours.

After incubation, the tissues were rinsed with phosphate buffered saline to remove any test materials and transferred to an assay medium containing 1 mg/ml of MTT and incubated for an additional 3±0.25 hours at 37±2° C. and 5±1% CO₂. Reduced MTT was subsequently extracted from the tissues and colorimetrically analyzed at 540 nm to determine tissue viability.

TABLE VI
MTT Assay

| Treatment | Viability |
|---|---|
| Non-UVB Exposed | 100 ± 1.4* |
| Untreated | 73 ± 6.6 |
| Mineral Oil | 74 ± 1.7 |
| WAF | 94 ± 8.1* |
| Sample B | 90 ± 1.8*, ** |
| Sample F | 92 ± 4.0*, ** |
| Sample A | 94 ± 2.9*, ** |
| 1 mM Trolox | 98 ± 5.6* |

*Significantly different from Untreated group (p < 0.05).
**Significantly different from Mineral Oil (p < 0.05).

Tissue viability in untreated and post-UVB mineral oil treated tissues resulted in an approximate 27% decrease in tissue viability. However, treatment with Sample B, Sample F, Sample A, or WAF immediately after UVB exposure prevented significant UVB induced decreases in tissue viability. Sample B treated tissue resulted in an approximate 10% reduction in tissue viability. Sample F resulted in an approximate 8% reduction in tissue viability. Sample A and WAF resulted in an approximate 6% reduction in tissue viability. Whole algal flour and algal oils effectively minimized UVB damage to epidermal tissue.

### Example 4

This method was designed to evaluate changes in tissue DNA thymine dimer content after exposure to UVB. The testing system used for this assay was MatTek's EpiDerm tissue, a skin model that consists of normal human-derived epidermal keratinocytes cultured to form a multilayered, highly differentiated model of the human epidermis. For this study, the tissues were exposed to UVB (300 mJ/cm²) and then treated topically for 24 hours with the test material. Following the treatment the DNA was extracted from the EpiDerm tissues and assayed for thymine dimer content. For the assay, samples of the DNA were immobilized on a solid membrane support and incubated with an antibody that recognizes thymine dimers in double stranded DNA. The primary antibody was then detected using a secondary antibody conjugated to a fluorescent dye. The membrane was then scanned using an excitation laser and emission filter combination appropriate for the fluorescent dye. With this method, the fluorescence intensity of each sample is proportional to the amount of the thymine dimers present in the sample.

Upon arrival, the tissues were stored at 4° C. until used. Prior to use, the tissues were removed from the agarose-shipping tray and placed into a 6-well plate containing 0.9 ml of assay medium. The tissues were allowed to incubate for at least 1 hour at 37±2° C. and 5±1% CO₂.

For use in this study 1 gram of WAF material, which is a powder, was combined with 10 ml of water and allowed mix on a rocking platform for one hour at room temperature. At the end of this mixing time the sample was centrifuged for 5 minutes to pellet any undissolved material and the supernatant (neat) was used for topical application. The remaining three materials (Sample B, Sample F and Sample A) were oil based materials and used as they were. For comparison purposes, mineral oil was also included in this study to serve as a control for the oil based materials to determine any non-specific effects due to the application of a general oil based material.

The tissues were subsequently exposed to 300 mJ/cm² UVB. UVB lamp intensity was measured using a UVX radiometer with a probe specific for UVB (detects 260-360 nm, max absorbance at 310 nm, calibrated at 310 nm) to determine exposure times required for the appropriate UVB dose. After the UVB exposure 100 μl of test material, mineral oil alone (oil control), 1 mM trolox (positive control), or PBS alone (negative control) was applied to the tissues and the tissues were incubated for 24 hours. At the end of the incubation period genomic DNA was recovered from the tissues.

Single tissues were placed into 1.5 ml centrifuge tubes containing 180 μl of Lysis Buffer One. After mincing the tissues with a pair of fine tipped scissors, 20 μl of Proteinase K was added to the tube and the tube was incubated overnight at 55±2° C. with occasional mixing/vortexing. After the Proteinase K digestion, 200 μl of Lysis Buffer Two was added to the tube and the tube was incubated at 70±2° C. for approximately 10 minutes. Next, the DNA was precipitated by the addition of 200 μl of 100% ethanol. The precipitated DNA was washed to remove cellular debris by applying the mixture to a DNEasy Spin Column and centrifuging the sample in a 2 ml collection tube at 8,000 RPM for 1 minute. The flow through and collection tube was discarded, and 500 μl of Wash Buffer One was added to the spin column and the column was placed into a new collection tube and centrifuged at 8,000 RPM for 1 minute. The flow through and collection tube was again discarded, and 500 μl of Wash Buffer Two was added to the spin column and the column was placed into a new collection tube and centrifuged at 14,000 RPM for 3 minutes. The spin column was then placed into a new 1.5 ml centrifuge tube and 110 μl of Elution Buffer was added to the column. The column was incubated for 1 minute at room temperature and then centrifuged at 8,000 RPM for 1 minute.

Extracted DNA was quantified via a fluorometric assay. A 10 μl aliquot of the DNA sample was mixed with 1.0 ml TE buffer and 100 μl of this diluted sample was transferred to a well in a 96-well plate. A series of DNA standards (0 to 500 ng/ml) was also transferred to wells in a 96-well plate in duplicate. Finally, 100 μl of dilute Cyquant Green dye was added to each well and the fluorescence intensity of each well was determined using an excitation wavelength of 480 nm and an emission wavelength of 520 nm.

Aliquots of genomic DNA samples or standards were converted to single stranded DNA by incubating the samples at 95° C. for 10 minutes and then chilled on ice. 100 μl or each sample or standard were then transferred to a DNA binding ELISA plate and incubated overnight at 4° C. On the following day the wells were rinsed once with 100 μl of PBS and then blocked with 150 μl of Assay Diluent for one hour at room temperature. After removing the Assay Diluent, 100 μl of anti-CPD antibody was added to each well and the plate was incubated for one hour at room temperature. After this incubation, the plate was washed three times with 250 μl of wash buffer per well, and then 150 μl of Blocking Reagent was added to the plate. The plate was blocked again for one hour at room temperature, and then washed three times as described before. 100 μl of Secondary Antibody was then added to each well and the plate was incubated for 1 hour at room temperature. After washing the plate again, 100 μl of substrate was added to each well and the plate was incubated for 5-20 minutes to allow for color generation in the plate. The color generation reaction was then stopped by the addition of 100 μl of stop solution and the plate was read at 460 nm using a plate reader.

TABLE VII
Thymine Dimer Formation

| Treatment ng/ml | Treatment ng/ml |
|---|---|
| Non-UVB Exposed | −16.4 ± 0.7*, ** |
| Untreated | 375.8 ± 9.9 |
| Mineral Oil | 334.3 ± 8.7 |
| WAF | 288.1 ± 11.1* |
| Sample B | 366.5 ± 28.6 |
| Sample F | 227.0 ± 13.2*, ** |
| Sample A | 260.9 ± 10.8*, ** |
| 1 mM Trolox | 296.5 ± 12.2* |

*Significantly different from Untreated group (p < 0.05)
**Significantly different from Mineral Oil group (p < 0.05)

In this study, irradiation of EpiDerm tissues with UVB light resulted in the formation of TT dimers within the genomic DNA. Post UVB treatment with mineral oil also resulted in a similar level of TT dimer formation within the genomic DNA. However, treatment with the three of the test material immediately after the UVB exposure resulted in a significant reduction in the amount of TT dimers formed. The three materials that were effective were the WAF, Sample F and Sample A materials. These results suggest that these test materials may be effective in preventing UVB induced DNA damage.

### Example 5

Using an eleven point scale, an Expert Sensory Evaluator assessed and compared 10 test oils. Several drops of each oil were placed on the volar forearm. After the olfactory profile was determined, the oil drops were gently rubbed into the forearm. In addition to the olfactory profile the following attributes were evaluated on a scale of 0-10 and tabulated: pre-absorption tackiness, post-absorption tackiness, absorbency, gloss, greasiness, silkiness, slipperiness, wetness, spreadability, and film residue. The oil remained on the forearm skin for approximately one hour.

TABLE VIII
Test Material Listing
Test Material Listing

| | |
|---|---|
| C14-2304.01 | Sample F |
| C14-2304.02 | Sample B |
| C14-2304.03 | Sample E |
| C14-2304.04 | Sample D |
| C14-2304.05 | Sample C |
| C14-2304.06 | Sample C FAME |
| C14-2304.07 | Oleic oil |
| C14-2304.08 | Argan oil |
| C14-2304.09 | High-oleic safflower oil |
| C14-2304.10 | Coconut oil |

TABLE IX
Test Material Attributes
Test Material Attributes

| Test | Tackiness- | Tackiness- | | | |
|---|---|---|---|---|---|
| Materials | Pre | Post | Olfactory | Absorbency | Gloss |
| C14-2304.01 | 1 | 3 | 1 | 4 | 1 |
| C14-2304.02 | 2 | 1 | 2 | 5 | 1 |
| C14-2304.03 | 6 | 4 | 4 | 3 | 0 |
| C14-2304.04 | 2 | 5 | 6 | 3 | 2 |
| C14-2304.05 | 0 | 1 | 1 | 5 | 1 |
| C14-2304.06 | 0 | 0 | 1 | 7 | 1 |
| C14-2304.07 | 0 | 0 | 1 | 7 | 1 |
| C14-2304.08 | 3 | 5 | 2 | 4 | 0 |
| C14-2304.09 | 2 | 1 | 4 | 8 | 1 |
| C14-2304.10 | 1 | 1 | 1 | 6 | 2 |
| Mean | 1.7 | 2.1 | 2.3 | 5.2 | 1.0 |
| Standard | 1.8 | 2.0 | 1.8 | 1.8 | 0.7 |
| Deviation | | | | | |

| Test | Film | Greas- | Silk- | Slipper- | Wet- | Spread- |
|---|---|---|---|---|---|---|
| Materials | Residue | iness | iness | iness | ness | ability |
| C14-2304.01 | 3 | 3 | 6 | 6 | 1 | 8 |
| C14-2304.02 | 3 | 5 | 5 | 5 | 1 | 6 |
| C14-2304.03 | 3 | 8 | 1 | 1 | 0 | 4 |
| C14-2304.04 | 5 | 6 | 5 | 5 | 3 | 5 |
| C14-2304.05 | 2 | 4 | 7 | 4 | 2 | 7 |
| C14-2304.06 | 1 | 1 | 5 | 7 | 1 | 8 |
| C14-2304.07 | 1 | 0 | 6 | 3 | 0 | 9 |
| C14-2304.08 | 2 | 1 | 2 | 2 | 1 | 6 |
| C14-2304.09 | 1 | 4 | 3 | 3 | 0 | 8 |
| C14-2304.10 | 1 | 3 | 2 | 4 | 0 | 8 |
| Mean | 2.2 | 3.5 | 4.2 | 4.0 | 0.9 | 6.9 |
| Standard | 1.3 | 2.5 | 2.0 | 1.8 | 1.0 | 1.6 |
| Deviation | | | | | | |

### Example 6

Transepidermal water loss (TEWL) is a measure of skin barrier function. The evaporimeter probe has two sensors, which measures the vapor pressure gradient arising within the chamber and between the skin and the surrounding air.

TEWL was measured using DermaLab Evaporimeter (Cortex Technology, Hadsund, Denmark). An absence of change in TEWL at post-treatment intervals compared to baseline indicated the mildness of the treatment, in that it has not disturbed barrier function. Decreases in TEWL indicated an improvement in skin barrier function, such that less water was lost through the skin barrier. TEWL measurements were taken from designated treatment sites and the control sites at each measurement interval.

Changes in skin conductance, impedance or capacitance are used to study epidermal hydration in vivo. The measurement is made on the difference in dielectric constant; skin has a low dielectric constant and water has a high dielectric constant of 81. When skin is hydrated, conductance and capacitance increases and impedance decreases. The measuring capacitor shows changes in capacitance according to the moisture content of the tissue. A glass lamina separate the metallic tracks in the probe head from the skin in order to prevent current conduction in the tissue. An electric scatter field penetrated the skin during the measurement and the dielectricity is determined.

Corneometer CM 825 (Courage and Khazaka, Germany) was used to measure the electrical capacitance/hydration of the skin. Three replicate measurements were taken from the designated treatment sites and the control site at baseline and immediate post-treatment intervals. If one measurement was more than ±10 units from the other measurements this measurement was not included in the analysis.

Both hydration and TEWL studies were performed in the following manner. Three days prior to the start of the study, enrolled subject began the washout period. Subjects received a neutral soap bar to use for cleansing their forearms and legs for the washout period. Subjects were given specific instruction prohibiting the use of all personal care products on the tests sites (volar surface of forearms and outer surface of the lower legs) for the entire study duration except for those provided. The volar surface of the forearms and outer lower legs was gently wiped with a damp disposable washcloth and patter dry with a paper towel. 6 test sites on the volar surfaces of the forearms and 6 tests sites on the outer surfaces of the lower legs were marked. Each test site was 4 cm×4 cm with at least 3 cm separation between adjacent test sites. Test sites were placed at least 2 cm from the wrist/knee joint and at least 2 cm from elbow/ankle joints. Treated sites and untreated control site within each forearm was randomly assigned. 2 sites serves as controls, one on the forearm and one on the outer lower leg.

Next, subject equilibrated by remaining seated for a minimum of 20 minutes in a room maintained at approximately 22±2° C. and 40±10% relative humidity. Temperature and humidity was recorded during subject testing before and after equilibration, before each TEWL measurement, and before each Corneometer measurement.

Baseline measurements were taken for hydration and TEWL studies. Skin hydration measurements by Corneometer at treated sites and untreated sites were measured. Only subject with mean Corneometer measurements of ≦40 on each test site proceeded with the study. TEWL readings by Evaporimeter at treated sites and untreated control sites were subsequently taken.

Following baseline measurements, test product application on the designated treatment test sites were performed. Approximately 2 mg/cm² of each product was applied to the randomized treated test sites. Measures were taken to ensure that the majority of the test material was transferred to the skin. One randomized site on the forearm and one randomized site on the outer lower leg served as an untreated control site.

Subjects were not allowed to cover, wet or wipe the test sites until the 8-hour post-treatment evaluation was complete. At 10 minutes, 1 hour and 40 minutes, and 7 hours and 40 minutes post treatment, subjects equilibrated by remaining quietly seated for a minimum of 20 minutes in a room maintained at approximately 22±2° C. and 40±10% relative humidity. Measurements were taken at the 30 minute, 2 hour, and 8 hour marks were taken. The results are shown in table set X and XI below.

### Skin Hydration

TABLE X(a)
Safflower Oil

| Parameter | 30 Minutes | 2 Hour | 8 Hour |
|---|---|---|---|
| Control Mean % | 4.95% | 8.28% | 6.01% |
| Difference from | | | |
| Baseline | | | |
| Control % of Subjects | 50.00% | 70.00% | 50.00% |
| Improved | | | |
| Treated Mean % | 62.99% | 54.00% | 38.94% |
| Difference from | | | |
| Baseline | | | |
| Treated % of Subjects | 100% | 100% | 90.00% |
| Improved | | | |

TABLE X(b)
Sample F (low polyunsaturates)

| Parameter | 30 Minute | 2 Hour | 8 Hour |
|---|---|---|---|
| Control Mean % | −0.68% | 2.51% | 5.17% |
| Difference from | | | |
| Baseline | | | |
| Control % of Subjects | 60.00% | 60.00% | 60.00% |
| Improved | | | |
| Treated Mean % | 58.22% | 44.53% | 26.21% |
| Difference from | | | |
| Baseline | | | |
| Treated % of Subjects | 100% | 90.00% | 100% |
| Improved | | | |

TABLE X(c)
Argan

| Parameter | 30 Minute | 2 Hour | 8 Hour |
|---|---|---|---|
| Control Mean % | −1.97% | 2.00% | 4.32% |
| Difference from | | | |
| Baseline | | | |
| Control % of Subjects | 50.00% | 60.00% | 60.00% |
| Improved | | | |
| Treated Mean % | 48.52% | 51.48% | 29.44% |
| Difference from | | | |
| Baseline | | | |
| Treated % of Subjects | 100% | 100% | 100% |
| Improved | | | |

TABLE X(d)
Sample D (Myristic)

| Parameter | 30 Minute | 2 Hour | 8 Hour |
|---|---|---|---|
| Control Mean % | 5.83% | 8.87% | 6.15% |
| Difference from | | | |
| Baseline | | | |
| Control % of Subjects | 50.00% | 70.00% | 60.00% |
| Improved | | | |
| Treated Mean % | 30.64% | 18.48% | 12.23% |
| Difference from | | | |
| Baseline | | | |
| Treated % of Subjects | 90.00% | 90.00% | 80.00% |
| Improved | | | |

TABLE X(e)
SOS

| Parameter | 30 Minute | 2 Hour | 8 Hour |
|---|---|---|---|
| Control Mean % | −1.61% | 1.08% | 2.99% |
| Difference from | | | |
| Baseline | | | |
| Control % of Subjects | 50.00% | 50.00% | 50.00% |
| Improved | | | |
| Treated Mean % | −3.58% | 12.36% | 12.29% |
| Difference from | | | |
| Baseline | | | |
| Treated % of Subjects | 50.00% | 60.00% | 70.00% |
| Improved | | | |

TABLE X(f)
WAF

| Parameter | 30 Minute | 2 Hour | 8 Hour |
|---|---|---|---|
| Control Mean % | 3.81% | 4.91% | 5.90% |
| Difference from | | | |
| Baseline | | | |
| Control % of Subjects | 60.00% | 40.00% | 60.00% |
| Improved | | | |
| Treated Mean % | 4.62% | 1.28% | 2.32% |
| Difference from | | | |
| Baseline | | | |
| Treated % of Subjects | 70.00% | 50.00% | 70.00% |
| Improved | | | |

TABLE X(g)
Sample B

| Parameter | 30 Minute | 2 Hour | 8 Hour |
|---|---|---|---|
| Control Mean % | −2.68% | −0.80% | 4.46% |
| Difference from | | | |
| Baseline | | | |
| Control % of Subjects | 50.00% | 50.00% | 50.00% |
| Improved | | | |
| Treated Mean % | 39.96% | 35.71% | 27.90% |
| Difference from | | | |
| Baseline | | | |
| Treated % of Subjects | 90.00% | 90.00% | 80.00% |
| Improved | | | |

TABLE X(h)
Coconut Oil

| Parameter | 30 Minute | 2 Hour | 8 Hour |
|---|---|---|---|
| Control Mean % | 0.85% | 5.23% | 5.12% |
| Difference from | | | |
| Baseline | | | |
| Control % of Subjects | 60.00% | 70.00% | 60.00% |
| Improved | | | |
| Treated Mean % | 37.55% | 43.38% | 27.51% |
| Difference from | | | |
| Baseline | | | |
| Treated % of Subjects | 100% | 100% | 100% |
| Improved | | | |

TABLE X(i)
Methyl Oleate of Sample C

| Parameter | 30 Minute | 2 Hour | 8 Hour |
|---|---|---|---|
| Control Mean % | 2.65% | 5.96% | 6.88% |
| Difference from | | | |
| Baseline | | | |
| Control % of Subjects | 60.00% | 60.00% | 70.00% |
| Improved | | | |
| Treated Mean % | 42.49% | 27.60% | 6.91% |
| Difference from | | | |
| Baseline | | | |
| Treated % of Subjects | 100% | 90.00% | 80.00% |
| Improved | | | |

TABLE X(j)
Methyl laurate

| Parameter | 30 Minute | 2 Hour | 8 Hour |
|---|---|---|---|
| Control Mean % | 2.90% | 6.58% | 9.07% |
| Difference from | | | |
| Baseline | | | |
| Control % of Subjects | 60.00% | 70.00% | 80.00% |
| Improved | | | |
| Treated Mean % | 42.45% | 30.93% | 14.97% |
| Difference from | | | |
| Baseline | | | |
| Treated % of Subjects | 100% | 80.00% | 90.00% |
| Improved | | | |

### Transepidermal Water Loss

TABLE XI(a)
Safflower Oil

| Parameter | 30 Minute | 2 Hour | 8 Hour |
|---|---|---|---|
| Control Mean % | −14.21% | −13.72% | −15.70% |
| Difference from | | | |
| Baseline | | | |
| Control % of Subjects | 80.00% | 70.00% | 60.00% |
| Improved | | | |
| Treated Mean % | −20.00% | −16.57% | 0.19% |
| Difference from | | | |
| Baseline | | | |
| Treated % of Subjects | 100% | 90.00% | 50.00% |
| Improved | | | |

TABLE XI(b)
Sample F (low polyunsaturates)

| Parameter | 30 Minute | 2 Hour | 8 Hour |
|---|---|---|---|
| Control Mean % | −11.24% | −12.58% | −15.94% |
| Difference from | | | |
| Baseline | | | |
| Control % of Subjects | 60.00% | 60.00% | 70.00% |
| Improved | | | |
| Treated Mean % | −12.55% | −12.36% | −6.56% |
| Difference from | | | |
| Baseline | | | |
| Treated % of Subjects | 80.00% | 80.00% | 60.00% |
| Improved | | | |

TABLE XI(c)
Argan

| Parameter | 30 Minute | 2 Hour | 8 Hour |
|---|---|---|---|
| Control Mean % | −7.00% | −8.33% | −6.00% |
| Difference from | | | |
| Baseline | | | |
| Control % of Subjects | 40.00% | 50.00% | 70.00% |
| Improved | | | |
| Treated Mean % | −17.41% | −15.59% | 0.40% |
| Difference from | | | |
| Baseline | | | |
| Treated % of Subjects | 90.00% | 90.00% | 50.00% |
| Improved | | | |

TABLE XI(d)
Myristic

| Parameter | 30 Minute | 2 Hour | 8 Hour |
|---|---|---|---|
| Control Mean % | −9.08% | −14.59% | −19.94% |
| Difference from | | | |
| Baseline | | | |
| Control % of Subjects | 60.00% | 60.00% | 70.00% |
| Improved | | | |
| Treated Mean % | −16.09% | −20.07% | −21.80% |
| Difference from | | | |
| Baseline | | | |
| Treated % of Subjects | 80.00% | 80.00% | 80.00% |
| Improved | | | |

TABLE XI(e)
SOS

| Parameter | 30 Minute | 2 Hour | 8 Hour |
|---|---|---|---|
| Control Mean % | −11.24% | −12.58% | −15.94% |
| Difference from | | | |
| Baseline | | | |
| Control % of Subjects | 60.00% | 60.00% | 70.00% |
| Improved | | | |
| Treated Mean % | −12.55% | −12.36% | −6.56% |
| Difference from | | | |
| Baseline | | | |
| Treated % of Subjects | 80.00% | 80.00% | 60.00% |
| Improved | | | |

TABLE XI(f)
WAF

| Parameter | 30 Minute | 2 Hour | 8 Hour |
|---|---|---|---|
| Control Mean % | −7.00% | −8.33% | −6.00% |
| Difference from | | | |
| Baseline | | | |
| Control % of Subjects | 40.00% | 50.00% | 70.00% |
| Improved | | | |
| Treated Mean % | −7.40% | −19.73% | −13.28% |
| Difference from | | | |
| Baseline | | | |
| Treated % of Subjects | 40.00% | 70.00% | 50.00% |
| Improved | | | |

TABLE XI(g)
Sample B

| Parameter | 30 Minute | 2 Hour | 8 Hour |
|---|---|---|---|
| Control Mean % | −11.06% | −18.54% | −18.05% |
| Difference from | | | |
| Baseline | | | |
| Control % of Subjects | 60.00% | 60.00% | 70.00% |
| Improved | | | |
| Treated Mean % | −10.00% | −14.39% | −3.51% |
| Difference from | | | |
| Baseline | | | |
| Treated % of Subjects | 80.00% | 70.00% | 50.00% |
| Improved | | | |

TABLE XI(h)
Coconut Oil

| Parameter | 30 Minute | 2 Hour | 8 Hour |
|---|---|---|---|
| Control Mean % | −11.67% | −2.22% | −5.93% |
| Difference from | | | |
| Baseline | | | |
| Control % of Subjects | 40.00% | 40.00% | 70.00% |
| Improved | | | |
| Treated Mean % | −22.18% | −11.27% | −1.58% |
| Difference from | | | |
| Baseline | | | |
| Treated % of Subjects | 90.00% | 70.00% | 40.00% |
| Improved | | | |

TABLE XI(i)
Methyl Oleate of Sample G

| Parameter | 30 Minute | 2 Hour | 8 Hour |
|---|---|---|---|
| Control Mean % | −12.20% | −4.92% | −7.80% |
| Difference from | | | |
| Baseline | | | |
| Control % of Subjects | 40.00% | 50.00% | 70.00% |
| Improved | | | |
| Treated Mean % | −27.45% | −22.96% | −15.64% |
| Difference from | | | |
| Baseline | | | |
| Treated % of Subjects | 90.00% | 70.00% | 60.00% |
| Improved | | | |

TABLE XI(j)
Methyl Laurate

| Parameter | 30 Minute | 2 Hour | 8 Hour |
|---|---|---|---|
| Control Mean % | −15.60% | −16.97% | −13.46% |
| Difference from | | | |
| Baseline | | | |
| Control % of Subjects | 60.00% | 70.00% | 80.00% |
| Improved | | | |
| Treated Mean % | −18.89% | −19.20% | −6.81% |
| Difference from | | | |
| Baseline | | | |
| Treated % of Subjects | 90.00% | 80.00% | 40.00% |
| Improved | | | |

### Example 7

Various exfoliant compositions below were formulated containing whole microalgal cells prepared as described above in Example 1. The microalgal cells in the formulations are originally from UTEX 1435 (Prototheca moriformis) that were chemically mutagenized and selected for high oil production. The microalgal cells were sieved through a wire mesh to obtain the desired particle size. The cells were then mixed with microalgal oil or with a silicon elastomer as a base liquid/cream.

TABLE XII
Formulation A: Face scrub

| Component | Amount |
|---|---|
| Microalgal cells (particles less than 0.425 mm) | 10 to 50% |
| Sample F oil | to 100% |

TABLE XIII
Formulation B: Body scrub

| Component | Amount |
|---|---|
| Microalgal cells (particles between 0.425 mm and 1 mm) | 10 to 50% |
| Sample F oil | to 100% |

TABLE XIV
Formulation C: Face scrubbing serum

| Component | Amount |
|---|---|
| Dow Corning EL8051 | 70.00% |
| Methyl ester of Sample F oil | 27.25% |
| Microalgal cells (particles less than 0.425 mm) | 2.75% |

The exfoliant formulations were surprisingly found to have a gritty texture and were effective in cleaning and exfoliating the skin. Moreover, the formulations left a silky skin feel after removal from the skin.

### Example 8

Hair strength properties were evaluated by repeated grooming using a combing/brushing apparatus as described in Evans and Park (J. Cosmet. Sci., 61, 439-455; November/December 2010). Repeated grooming was performed on medium brown hair tresses (International Hair Importers & Products, Glendale, N.Y.) weighing approximately 3 g and measuring 8″ in length and 1″ in width. Hair tresses were pre-treated by bleaching with a 9% hydrogen peroxide solution at pH of 10.2 for 20 minutes at 40° C., followed by rinsing under an intellifaucet set at 40° C. with a controlled flow rate of 1.0 gallons per minute. Tresses were allowed to equilibrate at 60% relative humidity overnight. All tresses were treated with 0.5 ml of oil by syringe and distributed to ensure the full dosage is applied to the hair. Four hair tresses were brushed simultaneously 10,000 times, with the collected broken fiber fragments evaluated every 1000 strokes. Eight replicates of the brushing experiments were performed for statistical relevance. The percent reduction in breakage is calculated as 100×(1−Mean# Broken fibers of treatment/Mean # Broken Fibers of Control).

Results of the grooming experiments are shown in Table XV. Hair treated with the oils were found to have 84% less hair breakage than untreated hair.

TABLE XV
Broken hair fragments after 10,000 strokes

| Treatment | Runs | Mean | Std Dev | Std Err Mean |
|---|---|---|---|---|
| Control | 8 | 92.25 | 12.78 | 4.52 |
| Sample F oil | 8 | 15.38 | 2.26 | 0.8 |
| Sample H oil | 8 | 15.38 | 3.11 | 1.1 |

### Example 9

Hair shine properties were evaluated using a Samba device (Bossa Nova Technologies, Culver City, Calif.) to measure the ratio of polarized and non-polarized light as an indicator of specular and diffuse reflection. The measured values from this instrument can be used to calculate shine and can be expressed as hair luster according to known formulas. Five shine measurements were performed on each tress, with eight replicate tresses being used per sample. All experiments were performed on bleached hair prepared as in the above example but using 6% hydrogen peroxide solution. All tresses were treated with 0.5 ml of oil by syringe and distributed to ensure the full dosage is applied to the hair. Shine values are expressed as % Luster (Reich-Robbins). The percent increase in shine is calculated as 100×(Mean Luster Value of Treated Hair−Mean Luster Value of treated of Control)/Mean Luster Value of Control.

Results of the shine measurements are shown in Table XVI. Hair treated with the oils were found to have over 210% more shine than untreated hair.

TABLE XVI
Hair luster values

| Treatment | Runs | Mean | Std Dev | Std Err Mean |
|---|---|---|---|---|
| Control | 8 | 23.90 | 1.32 | 0.47 |
| Sample F oil | 8 | 74.74 | 4.33 | 1.53 |
| Sample H oil | 8 | 77.34 | 4.52 | 1.60 |

1-5. (canceled)

6. The method of claim 24, wherein the composition is a shampoo, a hair conditioner, a hair mask, a skin oil, or a hair oil.

7-13. (canceled)

14. The method of claim 24, wherein the microalgae is of the genus Prototheca, Auxenochlorella, Chlorella, or Parachlorella.

15. The method of claim 24, wherein the microalgae is of the species Prototheca moriformis.

16. The method of claim 24, wherein the microalgae is of the species Chlorella (Auxeochlorella) protothecoides.

17. The method of claim 24, wherein the microalgae is Chlorella (Auxeochlorella) protothecoides and the oil has a fatty acid profile of greater than 15% C16:0 and greater than 55% 18:1.

18. The method of claim 24, wherein the oil has a fatty acid profile of greater than 50%, 60%, 70%, or 80% combined C10:0 and C12:0.

19. The method of claim 24, wherein the oil has a fatty acid profile of greater than 60% C10:0 and C12:0 and greater than 10% C14:0.

20. The method of claim 24, wherein the oil has a fatty acid profile of greater than 40%, 45%, or 50% C14:0.

21. The method of claim 24, wherein the oil has a fatty acid profile of greater than 85% C18:1 and less than 3% polyunsaturates.

22. The method of claim 24, wherein the oil has a fatty acid profile of at least 70% SOS and no more than 4% trisaturates.

23. The method of claim 24, wherein the oil has a fatty acid profile of greater than 50% C18:0 and greater than 30% C18:1.

24. A method for treating hair, comprising applying to the hair an effective amount of a topical composition comprising a cell oil produced by a microalgae, wherein the composition when applied to hair increases one or more of shine, combability, hair strength, resistance to UV damage, resistance to pollution damage, resistance to moisture loss, or resistance to split ends.

25-38. (canceled)

39. The method of claim 24, wherein hair treated with the composition has one or more properties of:
a) reduced entanglements;
b) reduced snagging;
c) reduced frizz;
d) reduced split ends;
e) less force required for brushing or combing;
f) reduced breakage;
g) increased resistance to heat damage such as from blow drying or hair irons; or
h) increased shine
as compared to untreated hair.

40. The method of claim 39, wherein hair treated with the composition has at least a 65%, 70%, 75%, 80%, or 84% reduction in hair breakage compared to untreated hair when tested after 10,000 brush strokes.

41. The method of claim 39, wherein hair treated with the composition has at least 170%, 180%, 190%, 200%, or 210% more shine compared to untreated hair.

42. The method of claim 40, wherein hair treated with the composition at least a 70% reduction in hair breakage and at least 190% more shine compared to untreated hair.

43. The method of claim 40, wherein hair treated with the composition at least an 80% reduction in hair breakage and at least 210% more shine compared to untreated hair.

44. The composition of claim 24.

45-47. (canceled)
